SEQUENCE LISTING (27 sequences)

SEQ ID NO: 1 - DNA, 903 bp, Yarrowia lipolytica
tgagatagtgagtttggaagtagtgttgagagtggtgaatgaaaagatgtctgtgcagtgagaaaaggagggacatctggccgtatttatagattttcccgttcctagatcttcaaaatacagttgtattcagccgcagcgcaaagtacgcatctgcatcggcagtagcactaatgtcgcaaagaatgcggttagacaaagaaaaaaggcacagccgtggcacaaacttctcatatctccaatgattgactaagatttaggtggcgctaaagtaaacaatgtctttgtagaacttgctagaagggtctgaaagagaatggaaatgtgtaaaggtgtgaaggatagtaattgtacaagggtgtacgagaagcaactcaacctggactatgtggattcgattcacctacttttaacattcaactcgaactaatgtcattataagcgtgccactcaagtctctatccttctccattcttccaacgtctgtgtcaggtgcatcgtacatattgtagatctctcgtcaaagttccgaagtgtatattccaataggactccgaaaacgcacaagtccacatgatgctagacattcggactccgaaacactaggaggggctattattgtgagatatttcattattaatgccgtttggccgaatttagccccgtttcagtggtattttaccgcgcattactggagcattcgaggggtttatgctgctaaggtttcgcttgtatgcgagatgcgagattatcgctgcggctgaaataattagtaaaaaagtgaaaaattaccagattgcaatatgtgctcttcgcaacagcagcagcagatataaataggagacgagatccgcaattcggttgtcacacactcacacacacacacacacacacacacacata

SEQ ID NO: 2 - DNA, 903 bp, Yarrowia lipolytica
tatgtgtgtgtgtgtgtgtgtgtgtgtgtgagtgtgtgacaaccgaattgcggatctcgtctcctatttatatctgctgctgctgttgcgaagagcacatattgcaatctggtaatttttcacttttttactaattatttcagccgcagcgataatctcgcatctcgcatacaagcgaaaccttagcagcataaacccctcgaatgctccagtaatgcgcggtaaaataccactgaaacggggctaaattcggccaaacggcattaataatgaaatatctcacaataatagcccctcctagtgtttcggagtccgaatgtctagcatcatgtggacttgtgcgttttcggagtcctattggaatatacacttcggaactttgacgagagatctacaatatgtacgatgcacctgacacagacgttggaagaatggagaaggatagagacttgagtggcacgcttataatgacattagttcgagttgaatgttaaaagtaggtgaatcgaatccacatagtccaggttgagttgcttctcgtacacccttgtacaattactatccttcacacctttacacatttccattctctttcagacccttctagcaagttctacaaagacattgtttactttagcgccacctaaatcttagtcaatcattggagatatgagaagtttgtgccacggctgtgccttttttctttgtctaaccgcattctttgcgacattagtgctactgccgatgcagatgcgtactttgcgctgcggctgaatacaactgtattttgaagatctaggaacgggaaaatctataaatacggccagatgtccctccttttctcactgcacagacatcttttcattcaccactctcaacactacttccaaactcactatctca

SEQ ID NO: 3 - DNA, 889 bp, Yarrowia lipolytica
tgtgagtttgcaagtgtggagagtggtagatgagatgatttgtttctgtgtactgagaaaaggagggagatgtggccctatttatagattttttcagttcctagatttacaagatacagttttattcagccgcagcacaacgcacgcatctgcatcagcagtagcactaatgtcgcaaataatggggttagacaaagaaaatggcacagccgtggcacaaacctcgcatatcaccagtgattgacttagatttaggtggcgctaaggtatacaatgtctttgtggaacttgctagaagggtctgaaagagtatggtgtgtataaacatgtaaagcatagactgtgtataagggtgtacgagaaccatttcaacttggactatgtggattcgattcacctacttttgacactcgatttgaacacatttcataatgagcgttccactcaagtccctatccttctccattcttccaacgtctgtatcaggtgcatcgtagatctctcgtcaaagttgaaagtgaacatgtgagactcgaaaaacgcacgataaagtccacatgatgccagccatttggactccgaaacacttgaaggtgatattattgtcagatctgtcattattataccgcttgatccaatttaggtccatttcaatagtattttatggtgcatcactggaacattcgagcggtatatgctgctaaggtttcgcttgtatgcgagatgcgagattgtcgctgcaactgaaataattagtaaaaaaagtgaaaagttatcagatggcaaaatgtgctcgtcgcaacagcagcagtagatataaataggagatgaggtccatggtttgtctctctctctcacacacacacacacacacacaaccacaaccaca

SEQ ID NO: 4 - DNA, 896 bp, Yarrowia lipolytica
tgtggttgtggttgtgtgtgtgtgtgtgtgtgtgagagagagagacaaaccatggacctcatctcctatttatatctactgctgctgttgcgacgagcacattttgccatctgataacttttcactttttttactaattatttcagttgcagcgacaatctcgcatctcgcatacaagcgaaaccttagcagcatataccgctcgaatgttccagtgatgcaccataaaatactattgaaatggacctaaattggatcaagcggtataataatgacagatctgacaataatatcaccttcaagtgtttcggagtccaaatggctggcatcatgtggactttatcgtgcgtttttcgagtctcacatgttcactttcaactttgacgagagatctacgatgcacctgatacagacgttggaagaatggagaaggatagggacttgagtggaacgctcattatgaaatgtgttcaaatcgagtgtcaaaagtaggtgaatcgaatccacatagtccaagttgaaatggttctcgtacacccttatacacagtctatgctttacatgtttatacacaccatactctttcagacccttctagcaagttccacaaagacattgtataccttagcgccacctaaatctaagtcaatcactggtgatatgcgaggtttgtgccacggctgtgccattttctttgtctaaccccattatttgcgacattagtgctactgctgatgcagatgcgtgcgttgtgctgcggctgaataaaactgtatcttgtaaatctaggaactgaaaaaatctataaatagggccacatctccctccttttctcagtacacagaaacaaatcatctcatctaccactctccacacttgcaaactcacatttcaca

SEQ ID NO: 5 - DNA, 852 bp, Yarrowia lipolytica
tgtgatagtgtgtttgtgtgtaggtggtgttggagatgagatggtgtgtctgtgtactgagaaaaggagggataacgggccgtatttatagattttcccgttcctagatctacaagatacagttgtattcagccgcagcacaatgcacgcatgggcatggcagtagcactaatgtcgcaaagaatggggtcaaacaaagagagagacgcggctgtggcacaatgatctcatatctagaacattcgtaacagattagatggcgctatgtaaacaatgtctttatggaatggggaaagaagatgggggaagactgtagccaacgccgcaccaaaagtgacctcaactgacccatatacacgagactcacttgcttccagatccctccttgatcctcctatcggaaacgagctcacttcacatctaccagctcttctgcttcgcagatcacccccagtacctactccaagctcaaagtacattccagtcggactccgaaaaaacgcaatatactctaatatgaggctgtcggactccgaaacactaaacaatcgccatttttgtctgattcaatacatattaacaccaattctctctttaaatccctttcagctccttgatgggttctggaatattcgagcggattgtgctgctaaggtttccattgtatgcaaggtgcgagattatcgctgcggctgaagaaactcaaaatgtgaaagttcatgatatggcgaagagtgcgcccaagacgaatgccagcactggacaatataaatagaagatggaatccgccgttcaattcaatcacaaatacaaacactcacaaacactcacaaacactcacacacaaccaca

SEQ ID NO: 6 - DNA, 850 bp, Yarrowia lipolytica
tggttgtgtgtgagtgtttgtgagtgtttgtgagtgtttgtatttgtgattgaattgaacggcggattccatcttctatttatattgtccagtgctggcattcgtcttgggcgcactcttcgccatatcatgaactttcacattttgagtttcttcagccgcagcgataatctcgcaccttgcatacaatggaaaccttagcagcacaatccgctcgaatattccagaacccatcaaggagctgaaagggatttaaagagagaattggtgttaatatgtattgaatcagacaaaaatggcgattgtttagtgtttcggagtccgacagcctcatattagagtatattgcgttttttcggagtccgactggaatgtactttgagcttggagtaggtactgggggtgatctgcgaagcagaagagctggtagatgtgaagtgagctcgtttccgataggaggatcaaggagggatctggaagcaagtgagtctcgtgtatatgggtcagttgaggtcacttttggtgcggcgttggctacagtcttcccccatcttctttccccattccataaagacattgtttacatagcgccatctaatctgttacgaatgttctagatatgagatcattgtgccacagccgcgtctctctctttgtttgaccccattctttgcgacattagtgctactgccatgcccatgcgtgcattgtgctgcggctgaatacaactgtatcttgtagatctaggaacgggaaaatctataaatacggcccgttatccctccttttctcagtacacagacacaccatctcatctccaacaccacctacacacaaacacactatcaca

SEQ ID NO: 7 - DNA, 500 bp, Yarrowia lipolytica
ttgcgaggggagaccagacatggtggaagcacatgagggcggggggccgatgccggtgctggaaagtcgaggtttgcaagctgcatttcgacaaacttgcggttcagtcacactccaacaacctggtaactccacacaagccacaaatccgttgtaccgtttcagctacaccgccacaagtttttttcattttgagccacaacctggatttcacatcagacatgaactgattaggctctgtggggggggatggcacagataagcagttgatgtagcagaagagaggggttggggcggattctcgagactccatcctgttgttttatcacgtgacttgtgtacttgtgtgcttgtggctctttcactgcttccgttgcatttgagcccctccagttgacccagaccctccatggaacacgtctctaaacaaggtttagtttttccaaatgactgaatactccgaaatcagtcgcagagagacttaacacactccaccacaa

SEQ ID NO: 8 - DNA, 781 bp, Yarrowia lipolytica
gagacccccgaacaaatgtgccacacccttgccaaaatgacgaatacacggcgtcgcggccgggaatcgaactcttggcaccgccacaggagtgaaatttgaaatttgaaatttgaaaaataattcacattttgagtttcaataatatatcgatgaccctcccaaaagacccaagtcgagacgcaaaaaaacacccagacgacatggatgcggtcacgtgaccgcaaaaaccgccccggaaatccgtttgtgacgtgttcaattccatctctatgtttttctgcggtttctacgatgccgcaatggtggccaatgtgcgtttcactgccgtagtggctggaacaagccacagggggtcgtcgggccaatcagacggtccctgacatggttctgcgccctaacccgggaactctaacccccgtggtggcgcaatcgctgtcttcatgtgctttatctcacgtgacggctggaatctggcagaagacggagtatgtacattttgtcgttggtcacgttatccctaaaacgtggtgtttaaactggtcgaatgcttggcccagaacacaagaagaaaaaaacgagacaacttgatcagtttcaacgccacagcaagcttgtcttcactgtggttggtcttctccacgccacaagcaacacgtacatgtcaattacgtcagggtcttttaagttctgtggcttttgaaccagttataaagaaccaaccacccttttttcaaagctaatcaagacggggaaattttttttttgatatttttcgaca

SEQ ID NO: 9 - DNA, 599 bp, Yarrowia lipolytica
tctagaaattatatttttttctgcatatacatattcatttaatagaggagcaaagattcttatccccactcaagtaatttgttgctataactaccaacctggaccaaccgtacatagtcgtagcgattattacaacacacccgaaaacaccataaaaaccctgtaattcgatacattggaattcgatcagaaaacaatgtctcttactatcagtttcgatcactaaacctctctctttaccatttgtatatattcaactccttctggtatacacacatgcccacaccaactgtacacactcaacactgcggctcatatatgcaccatgtatgacgcaacgtctcagtcaccatctcagtgaaccagtatgtaggtgtgtggtgggttgtacccagacgtgttccagaatatatatataaagggccgatttctcgttttcaaacctctccaaactgcacaatccaaccaccaagagtcactcctcctctaatacactgtgactcttctgactcgggaatcaaactttttacagtcactttataggtgcagcagtatctcgatctcaggacaaccacagaatcaccacctactacgacaca

SEQ ID NO: 10 - DNA, 791 bp, Yarrowia lipolytica
gatcagaactaagctaacactacagtagaaacagaagagaagccacgagaggagaaaaggtcacgtgatccacgagataacaccctccctgatatgttacgtcacgtggcacattctatcccttctgtgcaatgtcacgtgcctgacctttctcttcgaccgagagtcctacttttacttgcagctttctcgccgcaccctctaaatttgctcgtcggtgcatttacgatgtacagttgcgaatctcgggtgattttttgctcacattttggcggtagagcacttctttccggtgcgaaatccgtcaaaatgcaagttcggggtatttgcgactatggtgatagttttggaagtgtttctgagtcaattactttgggattctccttctcattcccttttgtatatggtattctatggcatgtatcatggcttggagttgtctgaaactcaaaatacaccttgataaaatctgattatctttaacctaaaagaggtagatcggaagagtacgaagatagttttccggaacagctacggaaaacggtttgtgaatattaagggaagttggggaccatggttttcgtgtcacatgatctacaagttgtgtcccctgtgtgtgttttgtgtcctgcagtttctgtaacgcagttgtatgtacagtatgtacagtaccaggacatactcctaacgggcctcctcttgcacctgcacctggacgagcaaatttcgagcagatatcgagttaccatgcaaagatcggcgtatatatagacaggagcttgggcctatgt

SEQ ID NO: 11 - DNA, 408 bp, Yarrowia lipolytica
accaatgaccatccagtaaactcttcccagaaaccctggtcttggagatccgtccgagttgtatgagccctgtccctggtttttggcggtggccagaatgtcgtagaccatgttgtgagtgacttcgagggagtttgctgagaataggaacctcctggagctggtcgtaaggactccgataccacacactctttacagtttgctcaaagatgcatagtgttccgctagctagtaatagtataaatctattcgcacatagactcagattttccagctgaaacgagcaaatatcaaactcaaaaaagagacatcagcagctcataattccagtatttccatctcttttttattcaactccaacaccatttctcacacacaaaaatgagccacgaccacggaagcatggat

SEQ ID NO: 12 - DNA, 105 bp, Yarrowia lipolytica
ctgaggtgtctcacaagtgccgtgcagtcccgcccccacttgcttctctttgtgtgtagtgtacgtacattatcgagaccgttgttcccgcccacctcgatccgg

SEQ ID NO: 13 - protein, 21 aa, Thosea asigna virus
GSGEGRGSLLTCGDVEENPGP

SEQ ID NO: 14 - protein, 22 aa, Sus scrofa
GSGATNFSLLKQAGDVEENPGP

SEQ ID NO: 15 - DNA, 63 bp, Thosea asigna virus
ggctccggcgagggccgaggctccctgctgacctgcggcgacgtcgaggagaaccccggcccc

SEQ ID NO: 16 - DNA, 1026 bp, Mus musculus
aagcttatgttctggcccaccaagaaggacctgaagaccgctatggaggtcttcgctctcttccagtgggccctgtctgctctcgtcatcgtgaccaccgtcatcattgtgaacctgtacctcgtcgtgttcacctcctactggcccgtcaccgtgctgatgctcacctggctggccttcgactggaagacccctgagcgaggcggtcgacgattcacctgcgtgcgaaagtggcgactgtggaagcactactctgactacttccccctcaagatggtcaagaccaaggacatctcccccgaccgaaactacattctggtttgtcacccccacggcctcatggctcactcttgtttcggtcacttcgctaccgacaccaccggtttctccaagaccttccccggaattaccccctacatgctgaccctcggcgctttcttctgggtccccttcctgcgagactacgtgatgtctaccggttcctgctctgtctcccgatcctctatggacttcctgctcacccagaagggcaccggtaacatgctcgtggtggtggtgggaggcctcgccgagtgtcgatactctacccccggctccaccaccctgttcctcaagaagcgacagggattcgtccgaaccgctctgaagcacggcgtgtccctcatccctgcctacgctttcggagagaccgacctgtacgaccagcacattttcacccccggtggattcgtcaaccgattccagaagtggttccagaagatggtgcacatctacccctgtgccttctacggacgaggcctgaccaagaactcttggggtctgctcccctactcccagcccgtcaccaccgtggtgggagagcctctgcctctccccaagatcgagaacccctctgaggagattgtggccaagtaccacaccctgtacattgacgctctgcgaaagctcttcgaccagcacaagaccaagttcggcatctccgagacccaggagctcgtcattgtgtaagtcattgtgtaacccggg

SEQ ID NO: 17 - DNA, 729 bp, Tobacco etch virus
atgggcgagtctctgttcaagggccccagagactacaaccccatctcctccaccatctgtcatctgaccaacgagtccgatggtcacactacctccctctacggtatcggcttcggccccttcatcatcactaacaagcatctgtttcgacgaaataacggtactctgctcgtgcaatccctccacggcgtgttcaaagtcaagaacaccaccaccctccagcagcacctcattgacggccgagacatgatcatcattcgaatgcccaaggacttccccccctttccccagaagctcaagtttcgagagccccagcgagaggaacgaatctgtctggtcaccaccaacttccagaccaagtccatgtcctccatggtgtccgacacctcttgcactttcccctcctctgacggcatcttctggaagcactggatccagaccaaggacggtcagtgcggttctcctctggtctccactcgagacggcttcatcgtgggcatccactccgcctccaacttcaccaacaccaataactacttcacctccgtccccaagaacttcatggagctgctgactaaccaagaggcccagcagtgggtgtctggttggcgactcaacgccgattccgtgctctggggcggccacaaagtcttcatggtcaagcccgaggagccttttcagcccgtcaaggaggccacccagctcatgaacagacgacgacgacgataa

SEQ ID NO: 18 - DNA, 20 bp, Artificial Sequence
  primer for detecting ura3
tcctggaggcagaagaactt

SEQ ID NO: 19 - DNA, 20 bp, Artificial Sequence
  primer for detecting ura3
agcccttctgactcacgtat

SEQ ID NO: 20 - DNA, 122 bp, Yarrowia lipolytica
gtgagtttcagaggcagcagcaattgccacgggctttgagcacacggccgggtgtggtcccattcccatcgacacaagacgccacgtcatccgaccagcactttttgcagtactaaccgcag

SEQ ID NO: 21 - DNA, 792 bp, Rhodosporidium toruloides
cggcttgttctctcctgctctggtgggctggcctgacatgtaatgtgctccgccgcaagtccgtcgtcggtctcaattcgacgttgaaagggcatagcgcaaggaagaaccctctgcggacatgcagaattactggctcgcctgctccttcgtctactggaataagtcctgtctcgttaaagccccaacgtcgtttttcgacgtttgtaaggcgcaagaggtgctatgggctacgcaggaagctgagaggacatagaagtcgggggaggaacggcgcagagcggcagttgcggaagcatgaggaaagcgagacggtccagcatctgcagcgccaatccgcaatctcctggttgagcctgcaccggaagcgtcggaacagtatgcgcagagtcgaacgcaagtaagaaagacgcaccctcacactcgcttacttcgagccatacaacggatcaaagctgcgcgtatctcggcttgtaagggccggaaagcaacctcggagatggacacgtcacatcaccaacttatcgatctcggccgtcgacgtcgcagagagggcgagagaagcggtgaaggagggaaacaacccctcgagagcatgatccgaccgaatctgcagcgcaggaagccgttacaagcccgcctcgagcgcaggtcgggtccagccgggggacgaaacgcgcgaggctgattcgtgagcgaaggaagccgcatcgacaagttcgctcccctttgccctctttcccatcacccgttctcgccttacccgctcagaacaacaccagatcactcaca

SEQ ID NO: 22 - DNA, 380 bp, Rhodosporidium toruloides
tagacgcacctcctccagcttcacctgcttccaaccttttccaccgcctgcaaccgcactttcgcctcgttccttcggactcttgcggctgcgatgttgtccagcatcgacaggagctgctttactttcgcttgacctgcttgccacctggtgctcgcacgatgccatatatcgcgagggaggcgagagagcggagttggctggatgacgctcgctccggcttgcagctggttgttacggtgttgcaagaatttctgtgcagtttgtacgagtggccccgcgttgtggatgatgtcggttcggttggcacggccttgctcgctcgctctctcgttgctcctcgctcttcaccacttcacttctaacactaactagctaca

SEQ ID NO: 23 - DNA, 785 bp, Rhodosporidium toruloides
tagcggcatgtcccgtcactgtccgcgaggtctgtagaaggagaacgaggtcgatgacggtgcgtcgaggtcgaggaggacgagagaaccgcccgacgccgttggaagcctcgctcccggtccgagcgagtgtagacagagtcgaacagggacgcagcagccacagcgggacgcttgagcgatgagcgaggtcgtggcgtccgccaacttgctcgtcgccgtcgaacctggcttgggttcgttctcaccactcgctctcagctcttcttccacgtgagcttggtttgaacactgcttggcttttcctaagaccgtcggactcgtctcgcacggcgaagcggcggtgggaacgggtcggggacgaacggggcgaacgattccatcggatggagccgaaacacggcgacgacggcttgggactggctcggttgggttgatatggatacagagacgctggatcaggctatggatgaagttgcagacatcccagtgctgtgtggacgccatgctgacctcgccatcttctccccgtcgaccatccctcccgactcgccattcgcgctgatcccgcgtcggatcctgcctgttgcacctcttgcgtgagtcttacagcttctctacaccctgccgcggcatcgaggtaacccggatctttctccctcgcctttgccaacctccgtcagatttggacccccgctcaacaacttcgaccgaacccaccgcttttctcgcttttctcgacacgccagtcgctttcgcagacacctatccgcacctaagcaacc

SEQ ID NO: 24 - DNA, 370 bp, Rhodosporidium toruloides
tgcagtgcactctgttgctcgtatcatgtcccactcccttgtatccctcgagtcggtcgactcttccctggcgagtccaagcggaggaggtggtcgtcgcctgacccgctcggagtgcgccgctcgacttggccctgggagaacaagcctgtgtgagtctgtctagcctgtcagcgaatgcgccagacgagtgcaagcgggtgagcgaggtcgaccctgctcgtcactcgctcgtcgggtgcggccgcatcgttgaacttgcacttctcactcgcactcgctctggtacagctacagtcactcgcttactactctgcaggttcacagcaactcacccgtccaactcccaccctcccccgtgcagcccacc

SEQ ID NO: 25 - DNA, 3849 bp, Streptomyces lavendulae
atgactctgcaagaaacctctgtgctggaacccactctgcaaggcaccaccaccctccccggtctgctggctcaaagagtggccgagcatcccgaggctattgccgtggcctaccgagacgataaactgaccttccgagagctggcttctcgatctgccgctctggctgactatctggagcatctcggtgtgtctgccgatgattgcgtcggtctcttcgtcgaaccctctattgatctgatggtcggtgcttggggtatcctcaatgctggcgctgcctatctccctctgtctcccgagtaccccgaagacagactccgatacatgatcgagaactctgagaccaagattattctggctcagcaacgactggtctcccgactgagagagctggctcccaaagacgtgactattgtgaccctccgagagtccgaggccttcgtccgacccgagggcactgaggctcccgccgctagatctgctagacccgacaccctcgcctacgtcatctacacctctggctctaccggcaagcccaagggtgtgatgatcgagcaccgatccatcgtgaatcagctcggttggctgagagagacctatgccattgaccgatccaaggtcatcctccagaagacccctatgtccttcgatgctgcccagtgggagattctctcccccgctaacggtgccactgtggtgatgggtgctcccggcgtgtacgctgatcccgagggcctcatcgagactatcgtgaagcacaacgtcaccaccctccagtgtgtgcccactctgctccaaggcctcattgatactgagaagttccccgagtgcgtgtccctccagcaaatcttttctggcggcgaagctctgtctcgactgctggccatccagactacccaagagatgcccggccgagctctgatcaacgtgtacggccctaccgagaccactattaattcctcctccttccccgtcgatcccgccgatctggatgaaggcccccagtctatttccattggttcccccgtccacggcactacctaccatattctggacaaggagaccctcaagcccgtcggtgtcggtgagattggtgagctgtatatcggtggcatccagctggctcgaggctatctgcacagagacgatctcaccgccgaacgattcctcgagatcgagctcgaggaaggcgccgaacccgtccgactgtacaaaactggcgatctgggtcagtggaacaatgatggcaccgtgcagttcgctggccgagctgacaatcaagtgaagctccgaggctaccgagtcgagctcgacgagatctctctcgccatcgagaaccacgattgggtgcgaaacgccgccgtcatcgtcaagaatgacggtcgaactggctttcagaacctcatcgcttgcatcgagctgtccgagaaggaagccgccctcatggatcaaggtaatcacggctcccaccacgcttccaaaaagtccaagctccaagtcaaggcccagctgtctaaccccggtctgagagatgacgccgaactggccgccagacccgcctttgatctggagggtgccgaacccacccccgagcagagagctagagtgttcgcccgaaaaacctaccgattttacgagggtggcgctgtgactcaagccgatctgctgggtctgctcggcgccaccgtcactgctggttactccagaaaggctgctgacctcgcccccgctgagctcggccagatcctccgatggtttggccaatacatctccgaggaacgactgctgcctaagtacggttatgcctcccccggcgccctctacgccactcaaatgtactttgagctcgagggcgtcggtggcctcaagcccggctactactactatcagcccgtgagacaccagctcgtgctgatttccgagcgagaggccaccggtaaggctactgcccaaatccatttcatcggcaagaaatccggcatcgagcccgtctacaaaaacaatattctggaggtgctcgagattgagactggccacatggtcggtctgtttgaacagattctgcccgcctatggtctggatatccacgaccgagcttacgaacccgccgtcaaagatctgctggacgtggctgacgaggactactatctgggtacttttgagctcgtgcctcacgctggcgccagagacgatcaagccgaggtgtacgtccaaactcacggtggtaaggtggccggtctgcccgaaggtcagtatcgatacgaaaacggcgaactcacccgattttccgacgacatcgtgctgaagaagcatgtcattgccatcaaccaatctgtgtaccaagccgcttcctttggcatctccgtgtattccagagccgaggaggagtggctcaaatacattaccctcggcaagaaactccagcatctgatgatgaacggcctcaacctcggctttatgtcctccggttactcttctaagaccggcaaccctctccccgcctctcgacgaatggacgctgtcctcggtgccaatggcgtggactctgctcccatgtactttttcgtcggcggcagaatctccgatgagcaaattggccacgagggtatgcgagaagactccgtccacatgcgaggtcccgccgaactcattagagacgacctcgtgtccttcctccccgattatatgatccccaaccgagtggtggtctttgaccgactgcctctgtccgccaacggtaaaattgacgtgaaggctctggccgcttccgatcaagtcaatgctgagctcgtcgagcgaccttttgtcgccccccgaaccgaaaccgagaaagaaatcgccgccgtctgggagaaggctctgcgacgagaaaacgcctctgtccaagatgatttcttcgaatccggcggcaactctctgatcgccgtgggtctcgtcagagaactgaacgctcgactcggtgtctccctccctctccagtctgtgctggagtcccccaccatcgagaagctggccagacgactggaaagagaagtcgctcaagagtcctcccgattcgtgcgactccacgccgaaaccggcaaggcccgacccgtcatctgttggcccggtctgggcggttaccctatgaatctgcgatccctcgctggtgaaatcggcctcggcagatccttctacggcgtccagtcttatggcatcaacgagggcgaaactccctatgagaccatcaccgagatggccaaaaaagacattgaggctctgaaggaaattcagcccgccggcccttacactctgtggggttactcctttggcgctagagtcgctttcgaaaccgcttaccagctcgagcaagctggcgaaaaggtggacaatctgtttctgattgcccccggctcccctaaagtgcgagccgagaacggtaaggtgtggggccgagaggcctccttcgctaaccgaggctacaccactattctcttttccgtcttcaccggcaccatttctggtcccgatctggacagatgtctggaaactgtcactgacgaagcctccttcgccgagttcatctccgagctcaagggtatcgacgtcgatctggctagacgaatcatctccgtggtgggtcaaacctacgagtttgagtattctttccacgagctcgctgagcgaactctgcaagcccctatttccatcttcaaggccgtgggcgacgactactccttcctcgagaactcttctggctactctgccgaacctcccactgtcattgatctcgacgccgaccattactctctgctgagagaggacatcggcgagctggtcaagcacatccgatatctgctgggcgagtaa

SEQ ID NO: 26 - DNA, 675 bp, Bacillus subtilis
atgaagatctacggcatctacatggatcgacccctctctcaagaggagaacgagcgattcatgtccttcatctcccccgagaagcgagagaagtgccgacgattctaccacaaggaggacgctcaccgaactctgctcggtgacgtgctggtccgatccgtcatttccagacagtaccagctcgacaagtctgacatccgattttccacccaagagtacggcaagccttgcatccccgatctgcccgacgcccacttcaacatctcccactctggtcgatgggtgatctgcgccttcgattcccagcccattggcatcgacatcgaaaagaccaagcccatctctctggagatcgccaagcgattcttctctaagactgagtactccgatctcctcgccaaggacaaagacgagcagactgactacttctaccacctctggtccatgaaggagtccttcatcaagcaagagggtaagggtctgtctctccctctggactccttctccgtgcgactgcaccaagacggtcaagtctctatcgagctgcccgactcccactctccttgctacatcaaaacctacgaggtggaccccggctacaaaatggctgtgtgtgccgctcaccccgacttccccgaggacatcaccatggtgtcctacgaggagctgctctaa

SEQ ID NO: 27 - protein, 11 aa, Escherichia coli
AANDENYALAA

(19) United States
(12) Patent Application Publication
(10) Pub. No.: US 2023/0183722 A1
(43) Pub. Date: Jun. 15, 2023

(54) GENE EXPRESSION SYSTEM FOR RAPID CONSTRUCTION OF MULTIPLE-GENE PATHWAY IN OLEAGINOUS YEASTS

(71) Applicant: WASHINGTON STATE UNIVERSITY, Pullman, WA (US)

(72) Inventors: Xiaochao XIONG, Pullman, WA (US); Shulin CHEN, Pullman, WA (US)

(21) Appl. No.: 17/995,624

(22) PCT Filed: Apr. 7, 2021
(86) PCT No.: PCT/US2021/026202

§ 371 Date: Oct. 6, 2022

Related U.S. Application Data
(60) Provisional application No. 63/008,098, filed on Apr. 10, 2020.
(60) Provisional application No. 63/147,352, filed on Feb. 9, 2021.

(51) Int. Cl. C12N 15/81; C12N 9/48

(52) U.S. Cl. CPC C12N 15/815; C12N 9/48; C12N 2830/002

(57) ABSTRACT

This invention discloses a novel system and method for expressing multiple gene products in oleaginous yeasts including Yarrowia lipolytica and Rhodotorula toruloides. More particularly, the present disclosure provides novel promoters functional in Y. lipolytica which can be used for producing a broad range of bioproducts.

### CROSS REFERENCE TO RELATED APPLICATIONS

This application claims priority to U.S. Provisional Patent Application Nos. 63/008,098 and 63/147,352 filed on Apr. 10, 2020 and Feb. 9, 2021, respectively, the disclosures of which are expressly incorporated herein.

### GOVERNMENT RIGHTS

This invention was made with government support under Grant/Contract Numbers 2019-31100-06053, awarded by the United States Department of Agriculture, National Institute of Food and Agriculture. The government has certain rights in the invention.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 26 kilobytes ASCII (text) file named “335006_ST25,” created on Apr. 6, 2021.

### BACKGROUND OF THE DISCLOSURE

As a Generally Recognized As Safe (GRAS) organism, the non-conventional yeast Y. lipolytica has been widely used and metabolically engineered for production of a suite of renewable chemicals and oleochemicals including fatty alcohols, long-chain dicarboxylic acids, organic acids including succinic acid and citric acid, polyketide triacetic acid lactone (TAL), and the sweetener erythritol. Synthetic biology of Y. lipolytica further enabled the strains to produce valuable natural products including eicosapentaenoic acid (EPA), astaxanthin, and ionone. A set of genetic manipulation tools including auxotrophic selection markers, optimized GFP for targeted overexpression and fluorescent tagging, and Ku70-deleted strain with increased homologous recombination frequency have been developed. Because promoters are critical to control gene expression at optimal levels and at specific timing for metabolic engineering, characterization and engineering of native promoters has been carried out in Y. lipolytica. Various native promoters including P_{FBA1}, P_{TDH1}, P_{GPM1}, P_{TEF}, and P_{FBAIIN} have been characterized as constitutive promoters, and have been used in metabolic engineering of Y. lipolytica for production of different products. The activities of some of these promoters such as P_{TEF} can be enhanced by the addition of tandem copies of upstream activation sequences (UASs). In addition to constitutive promoters, the growth phase inducible promoter hp4d, n-alkane inducible promoter of cytochrome P450 gene (alk1), oleic acid or methyl oleate inducible promoters of lip2 and pox2 were characterized. However, activation of these inducible promoters requires dramatic changes of culture conditions by adding different carbon sources, mainly hydrophobic substrates, as inducers, and the activities of these promoters are repressed by glucose present in the media, hence limiting their wide applications.

In addition to inducible promoters, repressible promoters can also be used to regulate and control gene expression in Y. lipolytica for the production of different products. Specifically, instead of deleting genes, repressible promoters can be used to inhibit target gene expression by deactivating a repressible promoter through the use of specific chemical or environmental factors. Repressible promoters are very useful for metabolic engineering to control the metabolic flux, especially when the gene cannot be deleted due to the essential function of targeted gene related to cell viability. For example, the methionine-repressible promoter P_{MET}3 was used to inhibit the expression of squalene synthase and channel flux into biosynthesis of amorphadiene, the precursor of artemisinin, in S. cerevisiae. A panel of promoters including P_{THR1}, P_{MET3} and P_{SER1} have been characterized as repressible promoters in both S. cerevisiae and methylotrophic yeast Pichia pastoris. However, there are no published reports of repressible promoters for Y. lipolytica.

R. toruloides, also known as Rhodosporidium toruloides (anamorph, Rhodotorula glutinis) is another important oleaginous yeast, and it has attracted much attention due to high content of lipid yield, tolerance to inhibitory compounds present in hydrolysate of lignocellulosic biomass, and its capability of utilization of C5 sugars. Other than microbial lipid production, it has been genetically modified to produce fatty alcohol and blue pigment indigoidine. Several constitutive promoters including P_{PGI}, P_{PGK}, P_{FBA}, P_{TPI} and P_{GRD} were characterized from R. toruloides genome. Multi-chassis engineering of heterologous pathways can increase the chances for successful production of natural products, and a host-independent expression system would further enable rapid construction of in the different chassis organisms. However, there has been no report of a promoter that is functional in both Y. lipolytica and R. toruloides.

With the advancement of synthetic biology, sophisticated design and complicated engineering have been implemented to reconstitute artificial biological systems including expression of large protein complexes with 17 subunits, re-engineered bacterial microcompartment organelles such as CO₂-fixing carboxysome, and modular signal transduction system such as G protein-coupled receptor (GPCR) to signaling in the cells. Especially, both degradation and biosynthesis pathways involve multiple genes to accomplish the biochemical function. To allow Saccharomyces cerevisiae to produce a plant-derived alkaloid strictosidine, 21 foreign genes were expressed in the yeast strain. To discover and engineer natural product biosynthesis, biosynthetic gene clusters (BGCs) are refactored by expression of the genes of interests under the characterized regulatory parts in heterologous host. In bacteria, multiple genes could be organized as a synthetic operon and their expression could be readily tuned by ribosome binding sites (RBS). In contrast, to construct the pathway in eukaryotes, each gene in a BGS was cloned between the upstream (promoter) and downstream (transcriptional terminator) regions, and then the expression cassettes are introduced into the host. As a result, the new tools to express multiple genes are continuously required to more efficiently engineer eukaryotic cell factories.

To enable convenient expression of multiple genes in eukaryotes, the picornavirus' 2A peptide has been adopted in the model organism S. cerevisiae, methylotrophic yeast Pichia pastoris and fungus Aspergillus nidulans. With the known self-splicing 2A peptides, polycistronic genes can be translated into peptides and “cleaved” during translation. The 2A peptides from picornavirus were successfully used to express heterologous genes in various eukaryotic cells including fungi, plants, insects and mammals. However, the 2A peptides consisting of around 20 amino acids from different viruses including equine rhinitis A virus (E2A), human foot-and-mouth disease virus (F2A), porcine teschovirus-1 (P2A), and Thosea asigna virus (T2A) demonstrated distinct cleavage efficiencies, and the function of the 2A peptides was not been tested in oleaginous yeast Y. lipolytica. Furthermore, one of the major drawbacks by using 2A peptides is addition of the partially digested 2A peptide sequences to the C-terminus of the proteins, interfering with enzymatic activity. It was observed that the order of genes linked with 2A peptides in the polycistronic construct had a strong influence on the pathway productivity. Finally, construction of a polycistronic segment composed of all individual genes separated with 2A sequences is still laborious and time consuming.

Substantial progress has been made to establish a molecular toolbox for genetic manipulation of the important industrially relevant strains Y. lipolytica and R. toruloides, but nevertheless the developed expression system heretofore known suffers from a number of disadvantages and limitations.

(a) The characterized inducible promoters in Y. lipolytica are mainly responsive to the hydrophobic substrates such as supplement of oleic acid, but repressed by glucose in the media. The application of these promoters is limited because it requires dramatic changes of carbon source.

(b) Repressible promoters have been employed as an important tool to downregulate genes expression. However, there are no such repressible promoters reported in Y. lipolytica.

(c) To construct a universal gene expression system, it requires a promoter that functions across the different strains. Although a wide range of promoters have been characterized in the industrially relevant organisms Y. lipolytica and R. toruloides, no reporter has been identified to be functional in both Y. lipolytica and R. toruloides.

(d) Self-splicing 2A peptides have been used as a powerful tool to construct polycistronic transcripts for expression of multiple genes in eukaryotes, but their application has not been explored in Y. lipolytica.

(e) The partially digested 2A peptide sequences will be appended to the C-terminus of the proteins, so development of a reliable expression system has to eliminate the interference.

(f) It is a labor-intensive procedure to develop a polycistronic construct consisting of multiple genes mediated with 2A peptide sequences by using traditional cloning approaches. A new approach for seamless assembly of gene fragments consisting of 2A sequences can facile the construction of large polycistronic construct.

In accordance with the present disclosure, methods and compositions, including expression vectors for expressing multiple genes in oleaginous yeasts Yarrowia lipolytica and Rhodotorula toruloides is provided.

### SUMMARY

The present disclosure is directed to a novel system and method for preparing nucleic acid constructs that encode multiple genes to regulate enzymatic pathways of oleaginous yeasts including Yarrowia lipolytica and Rhodotorula toruloides. These oleaginous yeasts have emerged as novel microbial chassis for the production of a broad range of bioproducts by synthetic biology. However, the current tools available for the manipulation of oleaginous yeasts are not optimal.

In accordance with one embodiment of the present disclosure, six copper-inducible promoters with bidirectional functionality, and five repressible promoters were isolated from Y. lipolytica and are utilized in expression vectors. The two repressible promoters disclosed herein (SEQ ID NOs: 10-11) showed relatively high activity compared with a strong constitutive promoter under non-repressing condition but could be almost fully repressed by supplement of low content of Cu²⁺ in Y. lipolytica.

In accordance with one embodiment the Cu²⁺-inducible promoters disclosed herein, including the promoter sequences of SEQ ID NOs: 1-6, can be engineered to improve the strength of each respective promoter by operably linking a tandem of upstream activation sequences (UASs). Such an engineered promoter was successfully used to construct a more productive pathway for production of a novel high-value bioproduct, wax ester than both native Cu²⁺-inducible and constitutive promoters. A synthetic promoter that is functional in both Y. lipolytica and R. toruloides has been developed by modification of a native promoter R. toruloides (modified RtGPD; SEQ ID NO: 21). By use of “self-cleaving” 2A peptide sequence from picornavirus, an elaborate, yet easy-to-assemble vector system is disclosed herein to conveniently express multiple genes under the control of a single promoter. Altogether, these combined efforts result in the development of a novel genetic manipulation system for the convenient expression of multiple genes in both Y. lipolytica and R. toruloides without the need for host-dependent optimization. It is a powerful tool applicable for multi-gene expression in the selected microbial hosts. In accordance with one embodiment, novel inducible and repressible promoters are provided that are functional in Y. lipolytica. These include the Cu²⁺-inducible promoters comprising a sequence selected from SEQ ID NOs: 1-6, the amino acid repressible promoters comprising a sequence selected from SEQ ID NOs: 7-9 and the Cu²⁺-repressible promoters comprising a sequence selected from SEQ ID NOs: 10-11.

In accordance with one embodiment a transcription element is provided that comprises a promoter and a polylinker operably linked to the said promoter, such that when a coding sequence is inserted into the polylinker site via one of the endonuclease restriction sites of the polylinker, the coding sequence is operably linked to the promoter and capable of being transcribed by said promoter. In one embodiment the promoter comprises of a nucleic acid sequence selected from the group consisting of SEQ ID NO: 1 (P_{MT-1}), SEQ ID NO: 2 (P_{MT-2}), SEQ ID NO: 3 (P_{MT-3}), SEQ ID NO: 4 (P_{MT-4}), SEQ ID NO: 5 (P_{MT-5}), SEQ ID NO: 6 (P_{MT-6}), SEQ ID NO: 7 (P_{THR1}), SEQ ID NO: 8 (P_{MET3}), SEQ ID NO: 9 (P_{SER1}), SEQ ID NO: 10 (P_{CTR1}), and SEQ ID NO: 11 (P_{CTR2}) or a nucleic acid sequence having at least 95% sequence identity with a nucleic acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11 and the promoter is operably linked to a polylinker sequence. In one embodiment the transcription element further comprises additional regulatory elements required for the expression of a coding sequence inserted into the polylinker site, including upstream activating sequences, a ribosome binding site (RBS) (in yeasts, more often known as Kozak sequences), transcription termination sequences and polyadenylation recognition sequences.

In one embodiment the promoter of the transcription element is a Cu²⁺-inducible promoter comprising a sequence selected from the group consisting of SEQ ID NO: 1, SEQ ID NO: 2, SEQ ID NO: 3, SEQ ID NO: 4, SEQ ID NO: 5, and SEQ ID NO: 6, optionally wherein the inducible promoter has 1, 2, 3, 4, 5, 6, 7, 8, 9, 10, 11, 12, 13, 14, 15 or 16 UAS sequences located upstream in a tandem array and operably linked to said promoter sequence, optionally wherein said UAS sequence comprises the sequence of SEQ ID NO: 12.

In one embodiment the promoter of the transcription element is a repressible promoter comprising a sequence selected from the group consisting of SEQ ID NO: 7, SEQ ID NO: 8, SEQ ID NO: 9, SEQ ID NO: 10, and SEQ ID NO: 11, optionally wherein the repressible promoter comprises the sequence of SEQ ID NO: 10 or SEQ ID NO: 11.

In one embodiment the transcription element is formed as a plasmid and further comprises a selectable marker gene and origin of replication that functions in Y. lipolytica and R. toruloides and optionally a second origin of replication that functions in E. coli. The transcription element can further comprises a series of tandemly repeated 2A polypeptide coding nucleic acid sequences, each with its own unique restriction site preceding the 2A polypeptide coding nucleic acid sequences for the insertion of a coding sequence that operably links the coding sequence to the promoter of the transcription element and to its respective 2A polypeptide coding nucleic acid sequence. In one embodiment the 2A polypeptide coding nucleic acid sequence encodes a polypeptide comprising the sequence of GSGEGRGSLLTCGDVEENPGP (SEQ ID NO: 13) or GSGATNFSLLKQAGDVEENPGP (SEQ ID NO: 14), optionally wherein the 2A polypeptide coding nucleic acid sequence comprises the sequence of SEQ ID NO: 15. In one embodiment the transcription element further comprises a nucleic acid encoding a TEV peptidase, optionally wherein the gene encoding the TEV peptidase is regulated by an inducible promoter, optionally wherein the gene encoding the TEV peptidase is operably linked to an inducible promoter of the transcription element as part of a polycistronic coding region. Expression of the gene coding TEV allows for the removal of the partial 2A peptides attached to C-terminus of the proteins expressed by a polycistronic region operably linked to the transcription element promoter. This cleavage eliminates inference caused by the residual 2A polypeptide remaining after self-cleavage and increases reliability of the expression system.

The isolated and engineered promoters can be used as novel standard parts to facilitate metabolic engineering and synthetic biology of this important organism. In accordance with one embodiment the transcription elements disclosed herein are used to transform oleaginous yeasts Y. lipolytica and R. toruloides to engineer cells to produce desired products. Accordingly, the present invention encompasses host cells comprising any of the transcription elements disclosed herein wherein the inducible or repressible promoter is operably linked to a heterologous coding sequence. More particularly, the host cell is a Y. lipolytica or R. toruloides cell, and optionally the host cell is a Ku70-deleted strain. In this context, the present disclosure also encompasses a method and vector system for expression of multiple genes in oleaginous yeasts Y. lipolytica and R. toruloides in a reliable and convenient way. The unique approach embedded in the platform overcomes the technical challenges related to expression of multiple genes in Y. lipolytica and R. toruloides for construction of complicated pathway leading to biosynthesis of biofuels and natural products.

In accordance with the present disclosure a set of molecular biology tools is provided for genetic manipulation of a non-conventional yeast Yarrowia lipolytica. One embodiment of the present disclosure is directed to a toolbox kit that includes easy-to-assemble and well-characterized genetic units including markers, promoters, terminators, and other essential parts. The usability of this kit was validated by development of recombinant strains for production of multiple bio-based products. The procedures have been streamlined to allow for convenient, standardized and scalable genetic operation of Y. lipolytica by using the toolbox kit providing one-stop and comprehensive tools for genes expression, deletion and integration in Y. lipolytica.

### BRIEF DESCRIPTION OF THE DRAWINGS

FIGS. 1A and 1B are schematic representations of an expression element comprising a promoter operably linked to a polycistronic region comprising a series of unique endonuclease restriction sites (E1, E2, and E3 in FIG. 1A; and E1, E2, E3, E4, E5 and E6 in FIG. 1B) where a coding sequence for a gene product can be inserted. Each of E1-E6 is followed by a 2A coding sequence, optionally followed by a TEV gene. This novel combination of elements in a single vector allows for rapid construction of a multiple-gene pathway for both Y. lipolytica and R. toruloides.

FIG. 2 is a bar graph presenting data that shows the strength of six cloned promoters P_{MT-1} to P_{MT-6} (SEQ ID NOs: 1-6, respectively) with and without 0.2 mM Cu²⁺ induction. LacZ assays were implemented to quantify the strength of promoters by using the cells grown on synthetic media lacking leucine for five hours.

FIG. 3 is a bar graph presenting data that shows the strength of three promoters including P_{THR1} (SEQ ID NO: 7), P_{MET3} (SEQ ID NO: 8) and P_{SER1} (SEQ ID NO: 9), with and without addition of amino acids. The amino acids were L-threonine (Thr), L-valine (Val), L-methionine (Met), and L-serine (Ser) with final contents ranging from 0.5 to 10 mM.

FIG. 4 is a bar graph presenting data that shows the strength of two promoters including P_{CTR1} (SEQ ID NO: 10) and P_{CTR2} (SEQ ID NO: 11) with and without addition of Cu²⁺. The promoter P_{TEF} was used a control to compare the strength.

FIG. 5 is a bar graph presenting data that shows strength of the P_{MT-2} promoter with an increasing copy number of UASs (ranging from two to 48) added upstream of the P_{MT-2} promoter both in presence and absence of Cu²⁺.

FIG. 6 is a graph showing the strength of the native promoter P_{MT-2} and a modified (P_{MT-2}-UAS16) engineered by introducing 16-copies of UASs with addition of various concentrations of Cu²⁺.

FIG. 7 is a bar graph presenting data that shows contents of fatty alcohols (C16-C18) and WEs produced by recombinants using a P_{MT-2} promoter to drive expression of MmWS and grown on 40 g/L glucose for four days (see Example 5 for details).

FIG. 8 is a bar graph presenting data that shows the strength of four well-characterized promoters from R. toruloides including P_{PGK}, P_{FBA}, P_{TPI}, and P_{GPD} as measured in Y. lipolytica. The addition of 16-copies of UASs upstream of P_{TPI}, and P_{PGK} significantly enhanced the activity of the promoters in Y. lipolytica.

FIG. 9 is a schematic map of the developed expression vector, pYaliHex.

FIG. 10 is a schematic representation of one procedure for cloning of genes and assembly of a polycistronic construct.

FIGS. 11A-11C present data regarding the expression of GFP and Red Fluorescent Protein (RFP) in Y. lipolytica in constructs with and without a sequence coding a 2A peptide. FIG. 11A is a schematic representation of plasmid pF2 which encodes a GFP fusion of cellodextrin transporter (CDT1) from fungus Neurospora crassa without 2A peptide. FIG. 11B is a schematic representation of plasmid pSX30, which encodes a GFP fusion of cellodextrin transporter (CDT1) with an intervening 2A peptide. FIG. 11C is a graph presenting the growth performance of recombinants comprising plasmid pF2 (16.7 g/L) and recombinants comprising plasmid pSX30 (20 g/L) when grown on cellobiose.

FIG. 12 is a schematic drawing of expression vector pYLexp2. This vector contains the promoter tef1N, which is one of the most frequently used promoters for expression of genes in Y. lipolytica. The map shows the key features and their organization in pYLexp2 (See Table 1 for details).

FIG. 13 is a schematic drawing of plasmid pUra3lxop. This plasmid contains marker gene, ura3 flanked by loxP sites, with the loxP sites flanked by a first and second polylinker sequence. The first and second polylinkers allow for the insertion of nucleic acid sequence having homology to genomic sequences that allows for targeted insertion of plasmid elements into the genome. In addition, other sequences (such as an inducible or repressible promoter or a gene construct) can be inserted into the plasmid and bracketed by the nucleic acid sequence having homology to genomic sequences for insertion into the genome in a targeted manner. This plasmid represents one embodiment used for gene disruption and/or gene insertion in Y. lipolytica including for example Y. lipolytica ΔKu70 and its derivatives.

FIG. 14 provides a schematic representation of the process for deletion/replacement of a gene in Y. lipolytica. 5′ flanking and 3′ flanking sequences having high homology (90-100% sequence identity) to two different genomic sequences are positioned outside the loxP flanked sequence to affect insertion of the plasmid sequences located between the 5′ flanking and 3′ flanking sequences, which can include a selectable marker gene (e.g., Ura3) and other genes.

FIG. 15 is a schematic drawing representing the use of the promoters and expression vectors of the present invention to manipulate the expression of multiple genes to redirect the biosynthetic machinery of Y. lipolytica to produce Indigoidine. More particularly, Y. lipolytica can be transformed with an expression vector comprising a single bidirectional inducible promoter (e.g. a promoter comprising a sequence selected from SEQ ID NOs 1-6) to simultaneously induce the expression of a bspA and sfp coding sequences to produce an active holo-BspA enzyme. The two genes, including sfp from Bacillus subtilis and bpsA from S. lavendulae were synthesized according to Y. lipolytica's codons as designated in a SEQ ID NO: 25 for bspA) and SEQ ID NO: 26 for sfp. Furthermore, an expression vector comprising a repressible promoter of the present invention (e.g. a promoter of SEQ ID NO: 10 or 11 downregulated by Cu²⁺) can be operably linked to an 2-oxoglutarate dehydrogenase (ogdh1 or ogdh2) coding sequence and the expression of Ogdh can be downregulated to assist in the production of Indigoidine. In a further embodiment the construct encoding Ogdh can further include a sequence encoding an SsrA peptide tag that is added to the encoded Ogdh protein, allowing the synthesized protein to be targeted for degradation upon induced expression of the ClpXP proteasome which degrades proteins comprising a ssrA peptide consisting of 11 amino acids, AANDENYALAA (SEQ ID NO: 27), for tighter control of its expression (See FIG. 16).

FIG. 16 is a schematic drawing representing the use of the promoters and expression vectors of the present invention shut down the activity of a target gene via Cu²⁺ mediated induced and repressed promoter activity. The target gene is expressed under the control of a Cu²⁺ repressible promoter (e.g. SEQ ID NO: 10 (P_{CTR1}) from an expression vector that adds the ssrA peptide to the carboxy terminus of the protein product of the target gene. Two genes (clpX and clpP) are each placed under the control of two a Cu²⁺ inducible promoters (e.g., SEQ ID NO: 1 (P_{MT-1}) and SEQ ID NO: 2 (P_{MT-2}), respectively) wherein upon induction by Cu²⁺ produces assembly of the ClpXP protease which degrades proteins comprising a ssrA peptide. A cell comprising the constructs of FIG. 16 (as shown in FIG. 17) produces the target gene product in the absence of promoter activating/inhibitory amounts of Cu²⁺, however contact of the cell with stimulating amounts of Cu²⁺ not only stops new target protein from being synthesized but also eliminates target protein that has already been synthesized for tighter control of the target gene expression.

FIG. 17 is a map of expression vector ClpXP.

### DETAILED DESCRIPTION

### Definitions

Unless defined otherwise, all technical and scientific terms used herein have the same meanings as commonly understood by one of ordinary skill in the art to which this invention belongs.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein the terms “native” or “natural” define a condition found in nature. A “native DNA sequence” is a DNA sequence present in nature that was produced by natural means but not generated by genetic engineering (e.g., using molecular biology/transformation techniques).

The term “endogenous” as used herein, refers to a natural state. For example a molecule (such as a direct repeat sequence) endogenous to a cell is a molecule present in the cell as found in nature. A “native” compound is an endogenous compound that has not been modified from its natural state.

As used herein, the term “exogenous” refers to a molecule not present in the composition found in nature. A nucleic acid that is exogenous to a cell, or a cell's genome, is a nucleic acid that comprises a sequence that is not native to the cell/cell's genome.

As used herein term “heterologous” in the context of a nucleic acid sequence defines a non-native juxtapositioning of two or more nucleic acids. For example a heterologous promoter operably linked to a second nucleic acid defines a recombinant relationship where a promoter is linked to a sequence that the promoter is not linked to naturally. A heterologous promoter may be exogenous to the host cell or it may be endogenous to the host cell (i.e., a polynucleotide native to the host cell, but integrated into a non-native location as a result of genetic manipulation by recombinant DNA techniques).

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

As used herein, the term “operably linked” refers to two components that have been placed into a functional relationship with one another. The term, “operably linked,” when used in reference to a regulatory sequence and a coding sequence, means that the regulatory sequence affects the expression of the linked coding sequence.

“Regulatory sequences,” “regulatory elements”, or “control elements,” refer to nucleic acid sequences that influence the timing and level/amount of transcription, RNA processing or stability, or translation of the associated coding sequence. Regulatory sequences may include promoters; translation leader sequences; 5′ and 3′ untranslated regions, introns; enhancers; stem-loop structures; repressor binding sequences; transcriptional termination sequences; polyadenylation recognition sequences; etc. Particular regulatory sequences may be located upstream and/or downstream of a coding sequence operably linked thereto. Also, particular regulatory sequences operably linked to a coding sequence may be located on the associated complementary strand of a double-stranded nucleic acid molecule. Linking can be accomplished by ligation at convenient restriction sites, however, elements need not be contiguous to be operably linked.

“Promoter” refers to a DNA sequence that initiates transcription of a coding sequence operably linked to the promoter and produces an RNA. This RNA may encode a protein, or can have a function in and of itself, such as tRNA, mRNA, or rRNA. In general, a coding sequence is located 3′ to a promoter sequence. Promoters that cause a gene to be transcribed in most cell types at most times are referred to herein as “constitutive promoters”. Promoters that allow the selective transcription of a gene in specified cell types or in response to developmental or environmental cues are referred to herein as “inducible promoters.”

As used herein a “bidirectional promoter” is a promoter that simultaneously initiates transcription from both strands of the double stranded promoter sequence.

Bidirectional promoters can be situated between two adjacent genes coded on opposite strands, wherein the 5′ ends of the adjacent genes are oriented toward one another and operably linked to the bidirectional promoter to simultaneously transcribe two genes based on the activation of a single promoter.

As used herein a “polylinker” or multiple cloning site” are used interchangeably and define a short DNA sequence, typically less than 100 nucleotides, containing two or more different recognition sites for cleavage by restriction enzymes.

As used herein the term “sequence identity” describes the ratio of the number of matching residues between two sequences (i.e., a nucleic acid or protein sequence) being compared over the total number of residues being compared in the alignment. Calculations of sequence identity can be determined using any standard technique known to those skilled in the art including, for example using a BLAST™ based homology search using the NCBI BLAST™ software (version 2.2.23) run using the default parameter settings (Stephen F. Altschul et al (1997), “Gapped BLAST and PSI-BLAST: a new generation of protein database search programs”, Nucleic Acids Res. 25:3389-3402).

A “gene product” as defined herein is any product produced by the gene. For example the gene product can be the direct transcriptional product of a gene (e.g., mRNA, tRNA, rRNA, antisense RNA, interfering RNA, ribozyme, structural RNA or any other type of RNA) or a protein produced by translation of a mRNA. Gene expression can be influenced by external signals, for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

A “host cell” is a cell which has been transformed or transfected, or is capable of transformation or transfection by an exogenous polynucleotide sequence. A host cell that has been transformed or transfected may be more specifically referred to as a “recombinant host cell”.

An “auxotroph” is an organism that is incapable of synthesizing a particular organic compound necessary for growth. An “auxotrophic marker” as used herein defines a gene that encodes an organic compound necessary for growth that is missing or deficient in the auxotroph.

### EMBODIMENTS

The present disclosure is directed to a novel system and method for preparing nucleic acid constructs for the transformation of oleaginous yeasts including Yarrowia lipolytica and Rhodotorula toruloides. More particularly the expression vectors described herein can be used to simultaneously express multiple gene products in a controlled manner to alter or regulate enzymatic pathways of Yarrowia lipolytica and Rhodotorula toruloides to produce desired products.

In accordance with one embodiment, novel inducible and repressible promoters are provided that are functional in Y. lipolytica. These include the Cu²⁺-inducible promoters comprising a sequence selected from SEQ ID NOs: 1-6, the repressible promoters comprising a sequence selected from SEQ ID NOs: 7-9 and the Cu²⁺-repressible promoters comprising a sequence selected from SEQ ID NOs: 10-11. In one embodiment one or more of these promoters are present as part of an expression vector that is configured for the insertion of a coding sequence of interest that operably links one of the promoter sequences of SEQ ID NOs 1-11 to the coding sequence of interest. Such vectors when introduced into a Y. lipolytica host cell allows for expression of the coding sequence of interest under the control of the inducible or repressible promoter.

In accordance with one embodiment a transcription element is provided that comprises a promoter and a polylinker operably linked to the said promoter, such that when a coding sequence is inserted into the polylinker site via one of the endonuclease restriction sites of the polylinker, the coding sequence is operably linked to the promoter and capable of being transcribed by said promoter upon introduction into a Y. lipolytica host cell. In one embodiment the promoter comprises an 850 to 903 bp nucleic acid sequence comprising a sequence selected from the group consisting of SEQ ID NO: 1 (P_{MT-1}), SEQ ID NO: 2 (P_{MT-2}), SEQ ID NO: 3 (P_{MT-3}), SEQ ID NO: 4 (P_{MT-4}), SEQ ID NO: 5 (P_{MT-5}), SEQ ID NO: 6 (P_{MT-6}), SEQ ID NO: 7 (P_{THR1}), SEQ ID NO: 8 (P_{MET3}), SEQ ID NO: 9 (P_{SER1}), SEQ ID NO: 10 (P_{CTR1}), and SEQ ID NO: 11 (P_{CRT2}) or a nucleic acid sequence having at least 80, 85, 90, 95% or 99% sequence identity with a nucleic acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11, wherein a polylinker is operably linked to said promoter sequence, such that introduction of a coding sequence into the polylinker region places the coding sequence under the transcriptional control of the promoter. In one embodiment the transcription element further comprises additional regulatory elements required for the expression of a coding sequence inserted into the polylinker site, including for example upstream activating sequences, a ribosome binding site (RBS), translational start codon, termination sequences and polyadenylation recognition sequences. In one embodiment the transcription element is formed as a plasmid and further comprises a selectable maker gene and an origin of replication that is functional in the target host cell (e.g., an E. coli or Y. lipolytica host cell).

In accordance with one embodiment a transcription element is provided that comprises a bidirectional promoter and a first and second polylinker, wherein the first and second polylinkers are operably linked to the said promoter on opposite ends of the double stranded promoter, such that when a first coding sequence is inserted into the first polylinker site and a second coding sequence is inserted into the second polylinker site via one of the endonuclease restriction sites of the first and second polylinkers, the first and second coding sequences are both operably linked to the bidirectional promoter and are both simultaneously transcribed by said promoter upon introduction into a Y. lipolytica host cell and activation of the promoter. In one embodiment the bidirectional promoter is selected from one of three pairs of a nucleic acid sequence including SEQ ID NO: 1 (P_{MT-1}) and SEQ ID NO: 2 (P_{MT-2}), SEQ ID NO: 3 (P_{MT-3}) and SEQ ID NO: 4 (P_{MT-4}) or SEQ ID NO: 5 (P_{MT-5}) and SEQ ID NO: 6 (P_{MT-6}), or nucleic acid sequence having at least 95% or 99% sequence identity with a nucleic acid sequence selected from the group consisting of SEQ ID NO: 1, SEQ ID NO: 2, SEQ ID NO: 3, SEQ ID NO: 4, SEQ ID NO: 5 and SEQ ID NO: 6. In one embodiment the bidirectional promoter comprises SEQ ID NO: 1 (P_{MT-1}) and SEQ ID NO: 2 (P_{MT-2}), or comprises sequences having at least 95% or 99% sequence identity with SEQ ID NO: 1 (P_{MT-1}) and SEQ ID NO: 2 (P_{MT-2}).

In one embodiment the promoter of the transcription element is a Cu²⁺-inducible promoter comprising a sequence selected from the group consisting of SEQ ID NO: 1, SEQ ID NO: 2, SEQ ID NO: 3, SEQ ID NO: 4, SEQ ID NO: 5 and SEQ ID NO: 6 or a sequence having at least 95% sequence identity with a sequence selected from the group consisting of SEQ ID NO: 1, SEQ ID NO: 2, SEQ ID NO: 3, SEQ ID NO: 4, SEQ ID NO: 5 and SEQ ID NO: 6. In one embodiment the promoter of the transcription element is a Cu²⁺-inducible promoter comprising a sequence selected from the group consisting of SEQ ID NO: 1, SEQ ID NO: 2, SEQ ID NO: 4, and SEQ ID NO: 6 or a sequence having at least 95% sequence identity with a sequence selected from the group consisting of SEQ ID NO: 1, SEQ ID NO: 2, SEQ ID NO: 4, and SEQ ID NO: 6. In one embodiment the promoter of the transcription element is a Cu²⁺-inducible promoter comprising a sequence selected from the group consisting of SEQ ID NO: 2 and SEQ ID NO: 6 or a sequence having at least 95% sequence identity with SEQ ID NO: 2 or SEQ ID NO: 6. In one embodiment the inducible promoter has 1, 2, 3, 4, 5, 6, 7, 8, 9, 10, 11, 12, 13, 14, 15 or 16 upstream activating sequences (UAS) located upstream of the promoter sequence of SEQ ID NO: 1, 2, 3, 4, 5 or 6 in a tandem array and operably linked to said promoter sequence, optionally wherein said UAS sequence comprises the sequence of SEQ ID NO: 12.

In one embodiment the promoter of the transcription element is a repressible promoter comprising a sequence selected from the group consisting of SEQ ID NO: 7, SEQ ID NO: 8, SEQ ID NO: 9, SEQ ID NO: 10, and SEQ ID NO: 11, or a sequence having at least 80, 85, 90, 95 or 99% sequence identity to a sequence selected from the group consisting of SEQ ID NO: 7, SEQ ID NO: 8, SEQ ID NO: 9, SEQ ID NO: 10, and SEQ ID NO: 11, optionally linked to a polylinker. In one embodiment the promoter of the transcription element is a Cu²⁺-repressible promoter comprising a sequence selected from the group consisting of SEQ ID NO: 10, and SEQ ID NO: 11, or a sequence having at least 95% sequence identity to a sequence selected from the group consisting of SEQ ID NO: 10, and SEQ ID NO: 11. In one embodiment the repressible promoter comprises the sequence of SEQ ID NO: 10 or SEQ ID NO: 11 or a sequence having at least 95% sequence identity to a sequence selected from the group consisting of SEQ ID NO: 10, and SEQ ID NO: 11, operably linked to a polylinker. In one embodiment the repressible promoter comprises the sequence of SEQ ID NO: 7, SEQ ID NO: 8 or SEQ ID NO: 9, or a sequence having at least 95% sequence identity to a sequence selected from the group consisting of SEQ ID NO: 7, SEQ ID NO: 8 or SEQ ID NO: 9, operably linked to a polylinker.

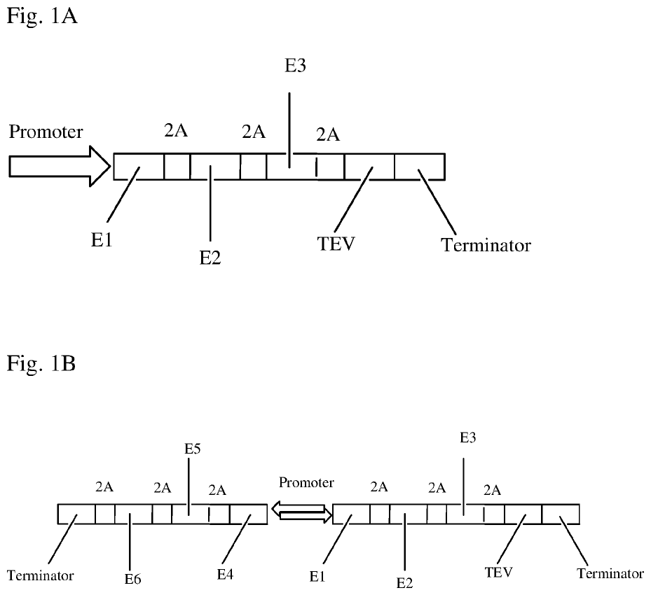

In one embodiment the transcription element is formed as a plasmid and further comprises a selectable marker gene and origin of replication that functions in Y. lipolytica and R. toruloides and optionally a second origin of replication that functions in E. coli. The transcription element can further comprises a series of tandemly repeated 2A polypeptide coding nucleic acid sequences, each with its own unique restriction site preceding the 2A polypeptide coding nucleic acid sequences to allow for the ease of inserting of a coding sequence of interest in operable linkage with the promoter of the transcription element and to its respective 2A polypeptide coding nucleic acid sequence. As shown in FIG. 1A one embodiment of a transcription element in accordance with the present disclosure comprises an inducible/repressible promoter (e.g., one comprising a sequence of SEQ ID NOs: 1-11) operably linked to a polycistronic region, wherein the polycistronic region comprises regions E1, E2 and E3 each representing one or more restrictions sites unique to the transcription element, each followed by a 2A protein coding sequence. Accordingly, using the unique restriction sites of E1, E2 and E3 the coding sequences of three separate genes can be introduced into the construction to be placed under the transcriptional control of the promoter and expressed with an attached 2A polypeptide.

In one embodiment the 2A polypeptide coding nucleic acid sequence encodes a polypeptide comprising the sequence of GSGEGRGSLLTCGDVEENPGP (SEQ ID NO: 13) or GSGATNFSLLKQAGDVEENPGP (SEQ ID NO: 14), optionally wherein the 2A polypeptide coding nucleic acid sequence comprises the sequence of SEQ ID NO: 15. In one embodiment the transcription element further comprises a nucleic acid encoding a TEV peptidase, optionally wherein the gene encoding the TEV peptidase is regulated by an inducible promoter, optionally wherein the gene encoding the TEV peptidase is operably linked to an inducible promoter of the transcription element as part of a polycistronic coding region (as shown in the embodiment of FIG. 1A). Expression of the gene encoding TEV allows for the removal of the partial 2A peptides attached to C-terminus of the proteins expressed by a polycistronic region operably linked to the transcription element promoter. This cleavage eliminates inference caused by the residual 2A polypeptide remaining after self-cleavage and release of the expressed polycistronic proteins, and increases the reliability of the expression system.

Alternatively, or in addition to the embodiment shown in FIG. 1A, since the Cu²⁺-inducible promoters represent three pairs of bidirectional promoters (SEQ ID NO: 1 (P_{MT-1}) and SEQ ID NO: 2 (P_{MT-2})); (SEQ ID NO: 3 (P_{MT-3}) and SEQ ID NO: 4 (P_{MT-4})); and (SEQ ID NO: 5 (P_{MT-5}) and SEQ ID NO: 6 (P_{MT-6})), transcription can simultaneously take place from each strand of the promoter of the transcription element. Therefore simultaneous transcription of two polycistronic regions can take place in the construct as shown in FIG. 1B.

In accordance with one embodiment any of the transcriptional elements disclosed herein further comprises one or more upstream activation sequences (UAS) located upstream of the promoter and operably linked to said promoter sequence. The tandemly repeated UAS elements can be identical or different and can range in number anywhere from 1 to 16. Optionally the UAS sequence may comprises the sequence of SEQ ID NO: 12 or a sequence having at least 95 or 99% sequence identity with SEQ ID NO: 12. In one embodiment 16 tandemly repeated UAS sequence comprises the sequence of SEQ ID NO: 12 are located upstream of the promoter wherein the promoter comprises a sequence selected from the group consisting of (SEQ ID NO: 1 (P_{MT-1}) and SEQ ID NO: 2 (P_{MT-2})); (SEQ ID NO: 3 (P_{MT-3}) and SEQ ID NO: 4 (P_{MT-4})); and (SEQ ID NO: 5 (P_{MT-5}) and SEQ ID NO: 6 (P_{MT-6})), or a sequence having at least 95% sequence identity to a sequence selected from the group consisting of (SEQ ID NO: 1 (P_{MT-1}) and SEQ ID NO: 2 (P_{MT-2})); (SEQ ID NO: 3 (P_{MT-3}) and SEQ ID NO: 4 (P_{MT-4})); and (SEQ ID NO: 5 (P_{MT-5}) and SEQ ID NO: 6 (P_{MT-6})). In accordance with one embodiment a transcription element is provided wherein the element comprises 1 to 16 tandemly repeated UAS sequence of SEQ ID NO: 12, or a sequence having at least 99% sequence identity with SEQ ID NO: 12, located upstream of the promoter, wherein the promoter comprises a sequence selected from the group consisting of (SEQ ID NO: 1 (P_{MT-1}) and SEQ ID NO: 2 (P_{MT-2})); (SEQ ID NO: 3 (P_{MT-3}) and SEQ ID NO: 4 (P_{MT-4})); and (SEQ ID NO: 5 (P_{MT-5}) and SEQ ID NO: 6 (P_{MT-6})), or a sequence having at least 95% sequence identity to a sequence selected from the group consisting of (SEQ ID NO: 1 (P_{MT-1}) and SEQ ID NO: 2 (P_{MT-2})); (SEQ ID NO: 3 (P_{MT-3}) and SEQ ID NO: 4 (P_{MT-4})); and (SEQ ID NO: 5 (P_{MT-5}) and SEQ ID NO: 6 (P_{MT-6})). In one embodiment a polylinker is operably linked to the promoter comprising the UAS sequences, and optionally further comprising one or more 2A polypeptide coding nucleic acid sequences located downstream from said polylinker, where each 2A polypeptide coding nucleic acid sequence is preceded by a unique endonuclease restriction site. In one embodiment the encoded 2A peptide has the sequence of GSGEGRGSLLTCGDVEENPGP (SEQ ID NO: 13) or GSGATNFSLLKQAGDVEENPGP (SEQ ID NO: 14), and optionally the 2A polypeptide coding nucleic acid sequence comprises the sequence of SEQ ID NO: 15 or a sequence having at least 95 or 99% sequence identity to SEQ ID NO: 15.

In one embodiment any of the transcription elements disclosed herein further comprises a ribosome binding site and an optional translation initiation codon positioned between said promoter and the polylinker. In a further embodiment any of the transcription elements disclosed herein further comprises an intron sequence located between the ribosome binding site and the polylinker of the transcription element, optionally wherein the intron comprises the 1st intron from the gene tef (SEQ ID NO: 20).

In accordance with one embodiment any of the transcription elements disclosed herein can be formed as a plasmid wherein the plasmid further comprises a selectable marker. In one embodiment the selectable marker is an auxotrophic marker, optionally wherein the auxotrophic marker is leu2 or ura 3. In one embodiment the selectable marker is an antibiotic resistance gene, including for example AmpR or TetR. In one embodiment the plasmid comprising the transcription element further comprises one or more origin of replication that allows the plasmid to replicate in the host organism. In one embodiment the plasmid comprises a replication region for Y. lipolytica and/or E. coli.

The transcription element as disclosed herein can be further combined with any of the elements disclosed in Tables 1-3. In one embodiment a coding sequence for a desired gene product is inserted into any of the transcription elements disclosed herein to operably link the promoters of SEQ ID NOs 1-11 to a heterologous coding sequence. The construct is then introduced into a host cell to modify the expression pattern of genes encoded by the host cell. In one embodiment the heterologous coding sequence is endogenous to the host cell, but the heterologous coding sequence is not naturally operably linked to the promoter of the transcription element. In one embodiment the heterologous coding sequence is not native to the host cell and represents an exogenous sequence. In one embodiment the host cell is a Yarrowia lipolytica or Rhodotorula toruloides host cell. In one embodiment the host cell is Y. lipolytica and optionally a Ku70-deleted strain of. lipolytica.

In one embodiment a method is provided for simultaneously inducing or repressing the expression of two gene products by inducing/repressing a single control element. In accordance with one embodiment a method of simultaneously inducing two or more coding regions from a single promoter comprise providing a host cell that comprises a Cu²⁺-inducible bidirectional promoter operably linked to both a first coding region on the plus strand of said promoter and a second coding region on the negative strand, wherein said promoter comprises a pair of nucleic acid sequences selected from the group of paired sequences consisting of SEQ ID NO: 1 and SEQ ID NO: 2, SEQ ID NO: 3 and SEQ ID NO: 4, and SEQ ID NO: 5, and SEQ ID NO: 6, or a sequence having at least 95% sequence identity to a nucleic acid sequence selected from the group consisting of SEQ ID NO: 1, SEQ ID NO: 2, SEQ ID NO: 3, SEQ ID NO: 4, SEQ ID NO: 5, and SEQ ID NO: 6; and contacting the host cell with an amount of Cu²⁺ that induces bidirectional transcription from said promoter to induce expression of said first and second coding regions. In one embodiment a plurality of genes are operably linked to said promoter in a tandem array wherein a 2A polypeptide coding sequence is located at the 3′ terminus of all but the last of said plurality of genes, optionally wherein the last encoded gene product is a TEV peptidase.

In one embodiment a method is provided for simultaneously repressing the expression of two or more genes from a single promoter comprise providing a host cell that comprises a Cu²⁺-inducible promoter operably linked to a polycistronic region coding multiple genes as disclosed in FIG. 1A wherein the coding sequences are separated by sequences coding 2A proteins, and optionally further comprising a TEV gene operably linked to the repressible promoter, wherein said promoter comprises of a nucleic acid sequence selected from the group consisting of SEQ ID NO: 1, SEQ ID NO: 2, SEQ ID NO: 3, SEQ ID NO: 4, SEQ ID NO: 5, and SEQ ID NO: 6, or a sequence having at least 95% sequence identity to a nucleic acid sequence selected from the group consisting of SEQ ID NO: 1, SEQ ID NO: 2, SEQ ID NO: 3, SEQ ID NO: 4, SEQ ID NO: 5, and SEQ ID NO: 6; and contacting the host cell with an amount of Cu²⁺ that induces transcription from said promoter to induce expression of coding regions contained in the polycistronic region. In one embodiment the promoter comprises of a nucleic acid sequence selected from the group consisting of SEQ ID NO: 1, SEQ ID NO: 2 and SEQ ID NO: 6, or a sequence having at least 95% sequence identity to a nucleic acid sequence selected from the group consisting of SEQ ID NO: 1, SEQ ID NO: 2 and SEQ ID NO: 6

In one embodiment a method is provided for simultaneously repressing the expression of two or more genes from a single promoter comprise providing a host cell that comprises a Cu²⁺-repressible promoter operably linked to a polycistronic region encoding multiple genes as disclosed in FIG. 1A wherein the coding sequences are separated by sequences coding 2A proteins, and optionally further comprising a TEV gene operably linked to the repressible promoter, wherein said promoter comprises of a nucleic acid sequence selected from the group consisting of SEQ ID NO: 7, SEQ ID NO: 8, SEQ ID NO: 9, SEQ ID NO: 10, and SEQ ID NO: 11, or a sequence having at least 95% sequence identity to a nucleic acid sequence selected from the group consisting of SEQ ID NO: 7, SEQ ID NO: 8, SEQ ID NO: 9, SEQ ID NO: 10, and SEQ ID NO: 11, and contacting said host cell with an amount of Cu²⁺ or an amino acid that inhibits transcription from said promoter to repress the expression of the coding regions contained in the polycistronic region. In one embodiment the repressible promoter comprises of a sequence selected from the group consisting of SEQ ID NO: 10 (CTR1) and SEQ ID NO: 11 (CTR2) and the host cell is contacted with an amount of Cu²⁺ that inhibits transcription from said promoter.

In accordance with one embodiment constructs are provided for use in conjunction with the transcription elements of the present disclosure, wherein the supplemental constructs are designed for the insertion, deletion or replacement of Yarrowia lipolytica or Rhodotorula toruloides host sequences. The method comprises the use of vectors that comprise, or allow for the insertion of, sequences that have high homology to sequences endogenous to the host organism. Such constructs can be used to delete genomic sequences or disrupt target endogenous genes to make null mutants. By including additional sequences between two sets of nucleic acid sequence that share 95 to 100% sequence identity to host sequences, the supplemental constructs can be used to insert genes or portions of genes (i.e., any of the inducible of repressible promoters disclosed herein) into a target location of the host organism's DNA. In one embodiment an inducible promoter selected from any one of SEQ ID NOs 1-6 is inserted to replace the native promoter of the target gene and place the encoded product under the control of the inducible promoter. In one embodiment a repressible promoter, selected from any one of SEQ ID NOs 7-11, is inserted to replace the native promoter of the target gene and place the encoded product under the control of the repressible promoter. In one embodiment the construct comprises a gene construct comprising a promoter selected from any one of SEQ ID NOs 7-11 operably linked to sequence having an open reading frame (i.e., a coding sequence), wherein upon transformation of the host cell, the construct inserts the gene construct in its entirety into the host cell's DNA, optionally replacing or disabling the native gene.

In one embodiment the supplemental constructs further comprise a selectable marker also located between the two sequences sharing high sequence identity with host DNA to allow for the selection of host cells that have successfully completed the homologous recombination event. In a further embodiment the selectable marker gene can be flanked with loxP sites whereupon subsequent introduction of cre recombinase activity results in the removal of the selectable marker gene.

In accordance with one embodiment a supplemental construct is provided comprising a gene cassette, wherein the gene cassette comprises a selectable marker and a promoter sequence selected from the group consisting of SEQ ID NO: 1 (P_{MT-1}), SEQ ID NO: 2 (P_{MT-2}), SEQ ID NO: 3 (P_{MT-3}), SEQ ID NO: 4 (P_{MT-4}), SEQ ID NO: 5 (P_{MT-5}), SEQ ID NO: 6 (P_{MT-6}), SEQ ID NO: 7 (P_{THR1}), SEQ ID NO: 8 (P_{MET3}), SEQ ID NO: 9 (P_{SER1}), SEQ ID NO: 10 (P_{CTR1}), and SEQ ID NO: 11 (P_{CTR2}) or a nucleic acid sequence having at least 90, 95% or 99% sequence identity with a nucleic acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11, wherein said gene cassette is flanked on both sides of the cassette with two unique sets of polylinkers or with two different DNA sequences that share 95-100% sequence identity to DNA sequences contained in the host cell. In one embodiment the two different DNA sequences that share 95-100% sequence identity to DNA sequences contained in the host cell comprise 26s rDNA sequences. In one embodiment the selectable marker gene is flanked with loxP sites. In one embodiment the promoter sequence is located outside the region flanked by the loxP sites, but within the sequences bracket by the sequences sharing high sequence identity to host DNA, and is linked to a polylinker or to a gene coding sequence.

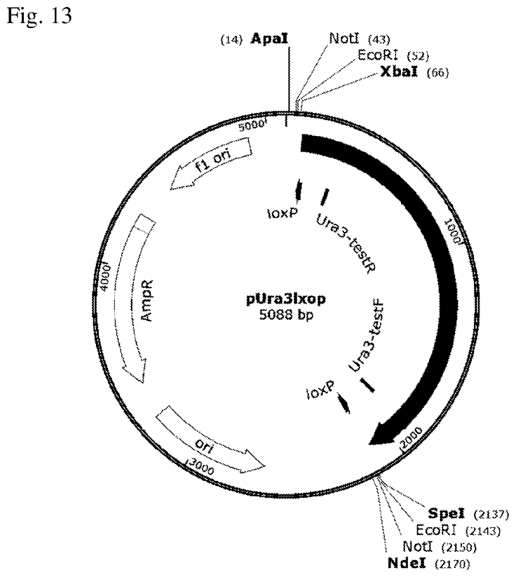

In accordance with one embodiment the experimental procedure of using the supplemental plasmids disclosed herein to disrupt a gene in Y. lipolytica and further remove the accompanying selectable marker (e.g., ura3) comprises the following steps. One vector suitable for use in such procedures is vector pURA3loxp as shown in FIG. 13. Using the unique restriction sites flanking the loxp sites of the vector, 5′ and 3′ sequences sharing high sequence identity with host sequences are inserted into the vector. These 5′ and 3′ are selected based on the target insertion site in the host and in one embodiment comprise 26s rDNA sequences. The plasmid is typically linearized and the host cell is transformed with the linearized plasmid. Cells comprising the desired recombination event are identified based on selection and verification by PCR techniques. Once cells comprising the desired recombination have been identified, the selectable marker can be subsequently removed by introducing cre recombinase activity into the recombinant host cell. In accordance with one embodiment the desired host transformant is transformed with a plasmid (e.g. pYKCre, see Table 2) to excise the sequences located between the loxp sites (including the selectable marker gene). Strains that have eliminated both the selectable marker and the cre expressing plasmid can then be selected.

In accordance with one embodiment kits are provided for manipulating Y. lipolytica cells. In accordance with one embodiment the kits include plasmids comprising the transcription elements disclosed herein and additional plasmid constructs for manipulating gene expression in Y. lipolytica, including any of the plasmids disclosed in Table 2.

In one embodiment, the expression vector, comprising any one of the promoters of SEQ ID NO: 1-11, that is included in the kit can have any of the other elements described herein, such as a selection marker, a cloning site, such as a multiple cloning site (i.e, a polylinker), an upstream activation site, an enhancer, a termination sequence, a signal peptide sequence, and the like. In another aspect, the expression vector can be a vector that replicates autonomously or integrates into the host cell genome. In another embodiment, the expression vector can be circularized or linearized (i.e., digested with a restriction enzyme so that a gene of interest can easily be cloned into the expression vector). In another embodiment, the kit can include an expression vector and a control ORF encoding a marker or control gene for expression (e.g., an ORF encoding a LacZ-alpha fragment) for use as a control to show that the expression vector is competent to be ligated and to be used with a gene of interest.

In another illustrative aspect, the kit can include other components for use with the expression vector, such as components for transformation of yeast cells, restriction enzymes for incorporating a protein coding sequence of interest into the expression vector, ligases, components for purification of expression vector constructs, buffers (e.g., a ligation buffer), instructions for use (e.g., to facilitate cloning), and any other components suitable for use in a kit for making and using the expression vectors described herein. In another embodiment, the expression vector or any other component of the kit can be included in the kit in a sealed tube (e.g., sterilized or not sterilized) or any other suitable container or package (e.g., sterilized or not sterilized). The kits described in the preceding paragraphs that include the expression vector comprising a promoter sequence selected form SEQ ID NOs: 1-11 can include a protein coding sequence operably linked to the promoter wherein the protein coding sequence is heterologous to the promoter (i.e., the combination does not occur in nature).

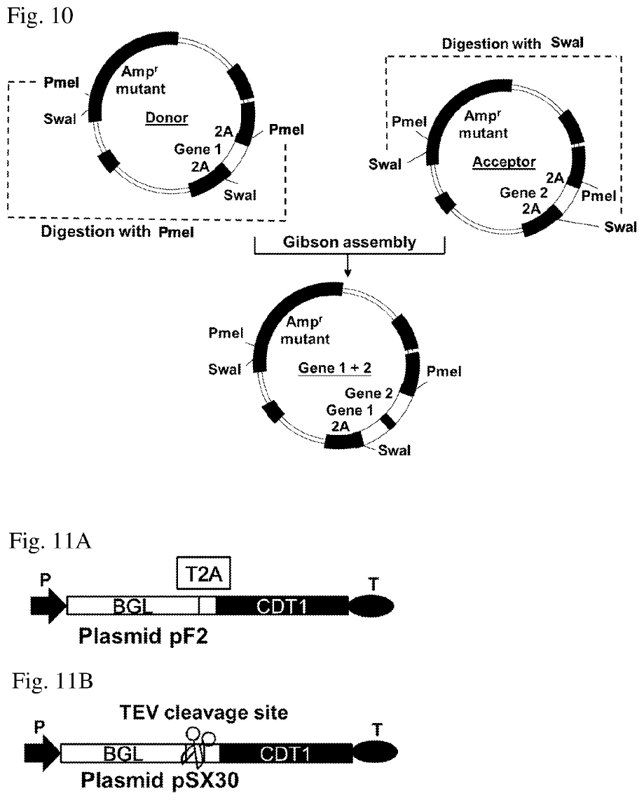

General cloning strategies including the procedures dependent on enzyme digestion and ligation and Gibson assembly can be employed to prepare the expression vectors disclosed herein as shown in FIG. 10. In one embodiment the transcription elements and expression vectors of the present disclosure include an ATG initiation codon located immediately prior to the polylinker. The expression vectors can be used for intracellular expression of a target gene or they can be integrated into the genome of the host cell. Typically the inserted coding sequence includes the translation termination codon, with TAA mostly commonly used in Y. lipolytica).

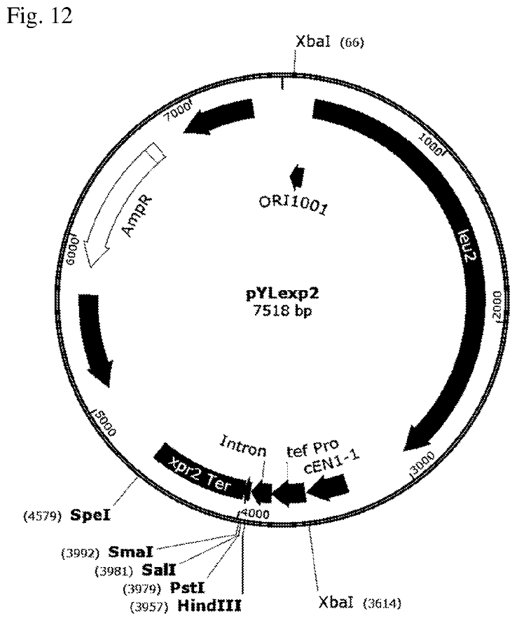

A gene of interest to be expressed can be inserted in to the expression vectors disclosed herein by introducing unique restriction site (e.g. AAGCTT for HindIII as listed in a polylinker before open reading frame (ORF)). Replication regions for Y. lipolytica including leu2, CEN1-1 and ORI1001 can be included in the expression vectors and can be removed by restriction sites flanking the origins of replication (see for example the use of XbaI digestion in FIG. 12). Similarly, the use of well-placed restriction sites can also be utilized for expression cassettes recovery and replacement, see for example FIG. 12 by XbaI/SpeI digestion and inserted into SpeI site of other expression vectors. Promoter and terminator sequences among different vectors can be swapped using the elements disclosed in Table 1.

In one embodiment a kit is provided comprising

a first plasmid comprising an inducible promoter sequence selected from the group consisting of SEQ ID NO: 1, SEQ ID NO: 2, SEQ ID NO: 3, SEQ ID NO: 4, SEQ ID NO: 5, and SEQ ID NO: 6, and a polylinker, wherein said polylinker is operably linked to said promoter; and

a second plasmid wherein said second plasmid comprises

a first and second pair of 34-bp loxp sites flanking a nucleic acid sequence encoding a selectable marker gene;

a first restriction site located upstream of said first loxp site; and

a second restriction site located downstream of said second loxp site, wherein said first and second restriction sites are different from each other and are unique to said second plasmid. In one embodiment the kit further comprises a repressible promoter selected from the group consisting of SEQ ID NO: 7, SEQ ID NO: 8, SEQ ID NO: 9, SEQ ID NO: 10, and SEQ ID NO: 11. In one embodiment the repressible promoter inserted into the second plasmid between the first and second restriction sites. In one embodiment the repressible promoter is formed as a third plasmid.

In one embodiment the second plasmid of the kit further comprises a nucleic acid sequence encoding a cre recombinase under the control of an inducible promoter. Alternatively, the kit can comprise a fourth plasmid wherein said fourth plasmid comprises a nucleic acid sequence encoding a cre recombinase. In one embodiment the second plasmid of the kit further comprises a first 26s rDNA sequence located upstream from said first restriction site and a second 26s rDNA sequence located downstream from said second restriction site.

In one embodiment a kit is provided comprising

a first plasmid comprising an inducible promoter sequence selected from the group consisting of SEQ ID NO: 1, SEQ ID NO: 2, SEQ ID NO: 3, SEQ ID NO: 4, SEQ ID NO: 5, and SEQ ID NO: 6, and a polylinker, wherein said polylinker is operably linked to said inducible promoter; and

a second plasmid wherein said second plasmid comprises

a repressible promoter selected from the group consisting of SEQ ID NO: 7, SEQ ID NO: 8, SEQ ID NO: 9, SEQ ID NO: 10, and SEQ ID NO: 11 and a polylinker, wherein said polylinker is operably linked to said repressible promoter. In one embodiment the second plasmid comprises an SsrA coding sequence located downstream of the polylinker such that when a coding sequence is inserted into the polylinker and the coding sequence is operably linked to the promoter, the protein expressed from said construct will comprise a C-terminal SsrA peptide tag. In a further embodiment the first plasmid of the kit further comprises a sequence, operably linked to the inducible promote, that encodes a protease that degrades an SsrA tagged protein. In one embodiment the nucleic acid sequences encoding the various subunits of the protease that degrades an SsrA tagged protein are under the control of a single inducible promoter. In another embodiment each of the nucleic acid sequences encoding the various subunits of the protease that degrades an SsrA tagged protein are under different inducible promoters. In one embodiment of this kit the inducible promoter(s) is selected from the group consisting of SEQ ID NO: 1, SEQ ID NO: 2, SEQ ID NO: 3, SEQ ID NO: 4, SEQ ID NO: 5 and SEQ ID NO: 6. In one embodiment the kit, the repressible promoter of the second plasmid is selected from the group consisting of SEQ ID NO: 10 and SEQ ID NO: 11.

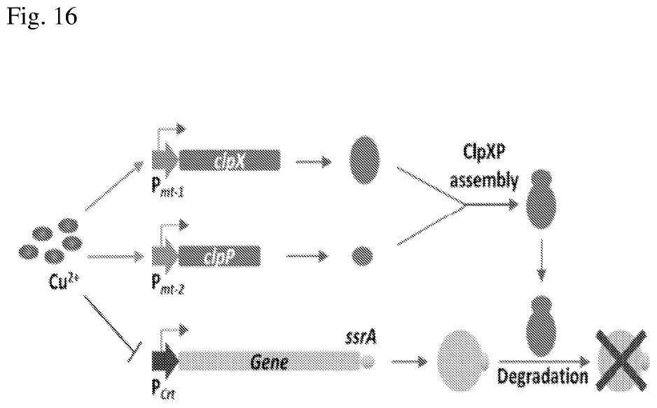

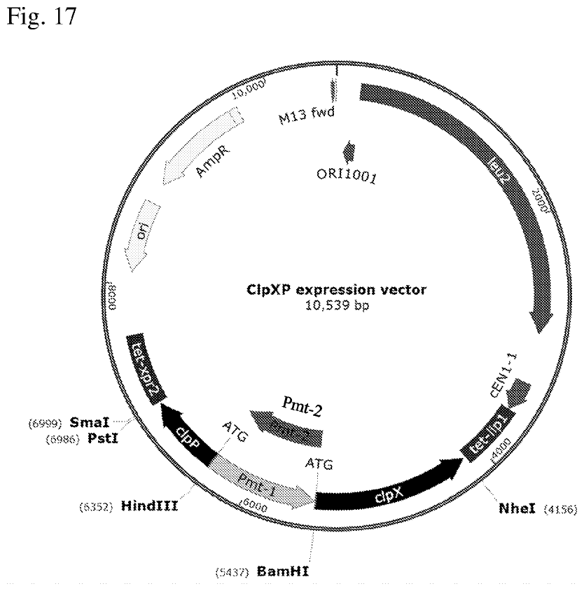

FIG. 16 provides a schematic drawing representing the use of the kit components to prepare a system where the promoters and expression vectors of the present invention are used to tightly regulate the expression of a target gene product and allow for rapidly turning off the activity of the target gene via Cu²⁺ mediated induced and repressed promoter activity. As shown in FIG. 16 copper-inducible promoters (Pmt-1/Pmt-2) drive the expression of genes clpX and clpP isolated from E. coli. In E. coli, ClpX and ClpP together form ClpXP proteasome, which can selectively recognizes and degrades the proteins comprising a C-terminus 11-amino-acid SsrA tag. The target gene is expressed under normal condition, whereas repressed with addition of copper. In one embodiment all four components have been engineered in one plasmid. The target gene is expressed under the control of a Cu²⁺ repressible promoter (e.g. SEQ ID NO: 10 (P_{CTR1}) or SEQ ID NO: 11 (P_{CTR2})) from an expression vector that adds the ssrA peptide to the carboxy terminus of the protein product of the target gene. Two genes (clpX and clpP) are each placed under the control of two a Cu²⁺ inducible promoters (e.g., SEQ ID NO: 1 (P_{MT-1}) and SEQ ID NO: 2 (P_{MT-2}), respectively, or any combination of inducible promoters selected from any of the Cu²⁺ inducible promoters of SEQ ID NOs: 1-6) wherein upon induction by Cu²⁺ produces assembly of the ClpXP protease which degrades proteins comprising a ssrA peptide. In one embodiment a bidirectional promoter comprising SEQ ID NO: 1 and SEQ ID NO: 2 is used to drive the expression of clpX and clpP off of opposite strands of the double stranded vector. In one embodiment clpX and clpP are expressed as part of a polycistronic construct operably linked to a promoter selected from the group consisting of SEQ ID NOs 1-6. A cell comprising the constructs of FIG. 16 produces the target gene product in the absence of promoter activing/inhibitory amounts of Cu²⁺, however contact of the cell with stimulating amounts of Cu²⁺ not only stops new target protein from being synthesized (by repressing expression of the target gene product) but also eliminates target protein that has already been synthesized (due to degradation of by the ClpXP protease) for tighter control of the target gene expression. Other degradation tags/protease combinations are known to those skilled in the art and are suitable for use in the present invention.

The kits of the present disclosure comprise elements necessary for the manipulation of gene expression in R. toruloides and Y. lipolytica. In particular, the present disclosure provides isolated genetic parts, method and vector systems. Six copper-inducible promoters with bidirectionality and five repressible promoters were isolated. Cu²⁺-repressible promoters showed relatively high activity compared with strong constitutive promoter under non-repressing condition but could be almost fully repressed by supplement of low content of Cu²⁺. One of the Cu²⁺-inducible promoters was engineered to improve the strength with tandem of upstream activation sequences (UASs). The utility and advantage of the engineered promoter were validated by production of a valuable bioproduct, wax ester with higher titer than both native Cu²⁺-inducible and constitutive promoters. A promoter was engineered to function across both R. toruloides and Y. lipolytica. By use of the self-splicing 2A peptides from picornavirus, it allowed expression of polycistronic genes in Y. lipolytica and R. toruloides. The gene encoding Tobacco Etch Virus (TEV) protease was further incorporated to remove the partial 2A peptides attached to C-terminus of the proteins expressed, eliminating the interfere with enzymatic activity. A vector system was developed for seamless assembly of a polycistronic construct spaced with 2A peptides. This invention provides a powerful biotechnology tool for expression of proteins, strain engineering and development, construction of complicated pathways, and building complex genetic network in oleaginous yeasts.

In accordance with one embodiment the novel promoters and expression vectors comprising such promoters can be used in applications for the pathway engineering of Y. lipolytica for biosynthesis of wax esters, indigoidine, building a system for more tightly controlled protein expression/degradation machinery, and extending the substrate range of the host to include cellobiose.

### Example 1

Identification of bidirectional copper-inducible promoters in Y. lipolytica

A Cu²⁺-inducible promoter P_{CUP1} has been identified in yeast S. cerevisiae, isolated from a gene encoding metallothionein, which is low molecular weight, cysteine-rich protein and capable of binding heavy metals such as copper, zinc, selenium, cadmium, mercury and silver. As disclosed herein, six genes namely MT-1 to MT-6 encoding metallothionein were retrieved in Y. lipolytica genome. These promoters are organized as three pairs (P_{MT-1} (SEQ ID NO: 1) and P_{MT-2} (SEQ ID NO: 2) located on opposing strands of DNA; (P_{MT-3} (SEQ ID NO: 3) and P_{MT-4} (SEQ ID NO: 4) located on opposing strands of DNA and (P_{MT-5} (SEQ ID NO: 5) and P_{MT-6} (SEQ ID NO: 6) located on opposing strands of DNA bidirectionalization to control expression of metallothionein in Y. lipolytica.

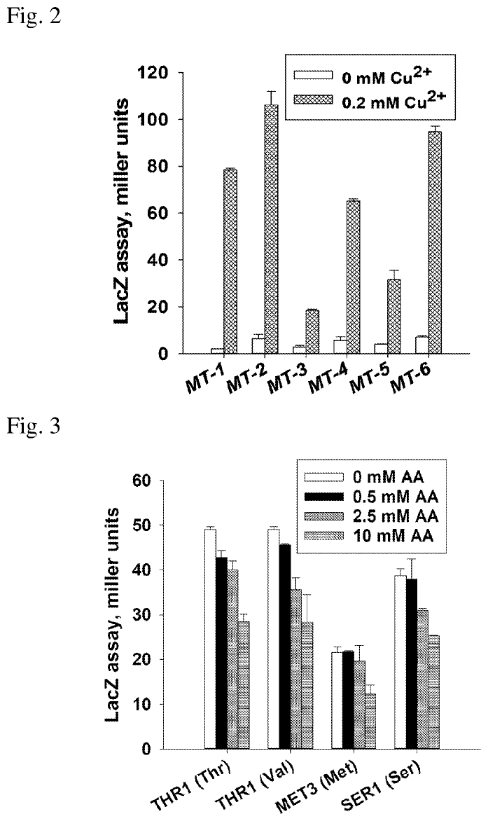

The strength of promoters, P_{MT-1} to P_{MT-6} was measured in presence of CuSO₄ by using β-galactosidase (LacZ). As shown in FIG. 2, the strength of all the selected promoters could be induced by CuSO₄ with a final concentration of 0.2 mM, which did not affect cell growth of Y. lipolytica, supplemented to the media. Among these promoters, in presence of Cu²⁺ P_{MT-2} had the highest strength, and the promoter with second highest activity was P_{MT-6}. More than 16-fold induction was achieved for P_{MT-2} by Cu²⁺. The strength of both P_{MT-2} and P_{MT-6} was comparable to that of constitutive promoters such as P_{TEF} identified previously, but they could be activated by Cu²⁺ as a cheap and efficient inducer. Especially, the promoters composed three pairs of bidirectional promoters including P_{MT-1}/P_{MT-2}, P_{MT-3}/P_{MT-4}, and P_{MT-5}/P_{MT-6}.

### Example 2

Identification of Amino Acids-Repressible Promoters in Y. lipolytica

To isolate the repressible promoters in Y. lipolytica, we checked the strength of promoters from genes THR1 (YALI0F13453p), MET3 (YALI0B08184p) and SER1 (YALI0F06468p) involved in amino acid biosynthesis with supplement of L-threonine or L-valine, L-methionine, and L-serine, respectively. The activities of P_{THR1} of P_{SER1} with addition of 10 mM amino acids were around half of their activities without supplement of amino acids for five hours (see FIG. 3). The strength of P_{MET3} maintained 66% with addition of 10 mM L-methionine compared with non-repressing conditions. The strength of these promoters could be inhibited by addition of the corresponding amino acids.

### Example 3

Identification of Copper-Repressible Promoters in Y. lipolytica

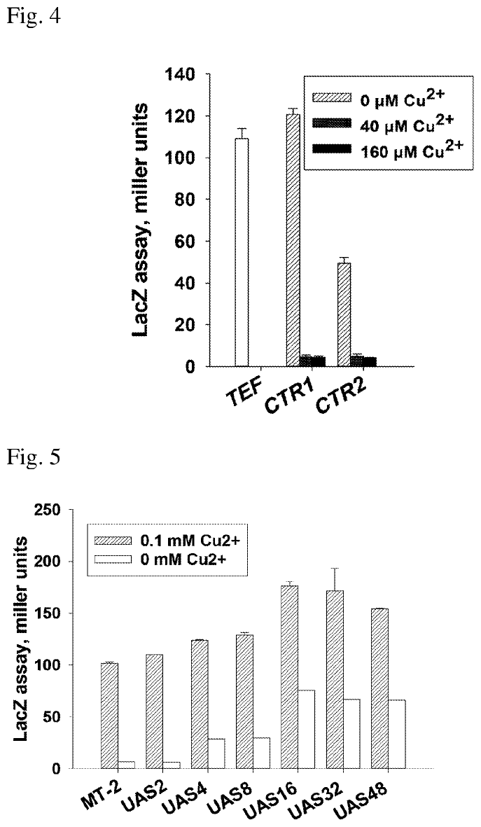

To seek repressible promoters responsive to cheaper chemical, two promoters of genes CTR1 (YALI0C20295p) and CTR2 (YALI0F24277p) belonging to copper transporter family were cloned and further investigated for their strength. As shown in FIG. 4, the strength of P_{CTR1} and P_{CTR2} was almost fully inhibited by low concentration of Cu²⁺. The strength of P_{CTR1} in presence of 0.16 mM Cu²⁺ was only 5% of the activity without addition of Cu²⁺. Furthermore, the strengthen of P_{CTR1} without repression was much higher than the strong promoter P_{TEF}, whereas the strengthen of P_{CTR2} without repression was a half of that of P_{TEF}.

### Example 4

Engineering of Hybrid Promoters Consisting of P_{MT-2} in Y. lipolytica

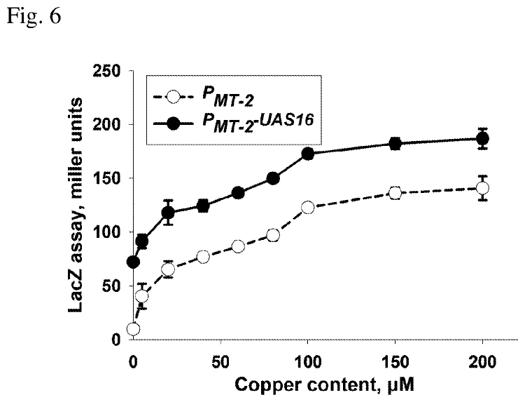

The effects of UAS copy number on the strength of P_{MT-2} with and without addition of copper were investigated (FIG. 5). The results showed that both the basal activity without copper induction and strength in presence of 0.1 mM CuSO₄ reached their highest level when 16 tandem repeats of UAS (UAS16) was added to upstream of P_{MT-2} Even without copper induction, P_{MT-2}-UAS16 had relatively high basal strength. It indicated that both P_{MT-2} and its hybrid form P_{MT-2}-UAS16 were induced with copper content up to 0.2 mM, and P_{MT-2}-UAS16 showed higher activities than P_{MT-2} under the same conditions (FIG. 6). By using tandem of UASs, it further increased dynamic regulation range of copper inducible promoters, hence providing additional benefits for control of genes expression and metabolic engineering in Y. lipolytica.

### Example 5

### Utility of Isolated and Engineered Promoters for Metabolic Engineering of Y. Lipolytica for Producing Wax Ester

To demonstrate the utility of promoters isolated and engineered in this study, we used the promoter P_{MT-2}-UAS16 to engineer a pathway for production of bio-based long-chain wax ester (WE). WEs are high-value products widely used for making personal cosmetics, pharmaceutical drugs and lubricants. In the past, WEs were obtained from whale oil; however, bans on hunting sperm whales now preclude its access for industrial markets. Current practices for WE production rely on jojoba oil from the shrub Simmondsia chinensis, which is adapted to arid areas such as the desert regions and is not suitable for large-scale growth. The limited availability and high production cost prevent use of WE in widespread applications. Microbial production of WEs provides an alternative route that can potentially overcome these obstacles and promote sustainable, large-scale and high-efficiency production of WEs. In our previous studies, we engineered Y. lipolytica to produce fatty alcohol (C16-C18) by expression of TaFAR gene encoding fatty acyl-CoA reductase from Barn owl (Tyto alba). In the present invention, we extended this fatty alcohol forming pathway to produce WEs by expression of codon-optimized MmWS gene SEQ ID NO: 16) from mouse (Mus musculus), which encodes WE synthase/acyl coenzyme A:diacylglycerol acyltransferase (WS/DGAT).

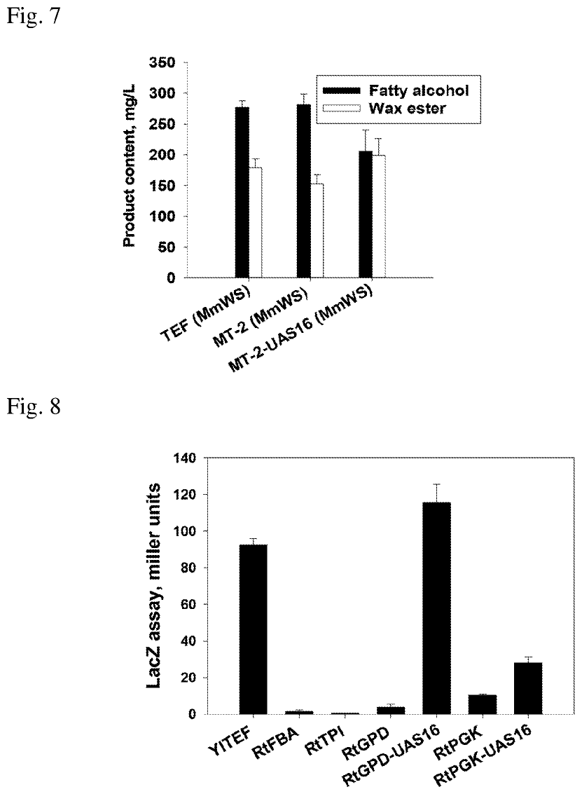

Gas chromatography (GC) analysis showed that the strain expressing MmWS under control of P_{MT-2}-UAS16 in presence of 0.2 mM CuSO₄ produced a metabolite, whose retention time matched that of the standard, palmityl palmitic acid (C16, C16). We further confirmed the structure of products including the other minor products including stearyl stearic acid (C18, C18) and palmitoleic stearic acid (C16:1, C18) by GC-MS. The titer of WEs produced by the recombinant grown on 40 g/L glucose for four days was up to 199.4 mg/L, which was higher than the titer of WEs at 179.6 mg/L produced by the fatty alcohol-producing strain expressing MmWS driven by P_{TEF}. Similarly, expression of MmWS by use of P_{MT-2} with 0.2 mM Cu²⁺ addition resulted in accumulation of 150.9 mg/L of WEs. There were still high contents of fatty alcohols produced by all the strains (FIG. 7). The first-time formation of long-chain WEs by engineering of an oleaginous yeast has been demonstrated in this study, and higher yield can be achieved by both pathway engineering and fermentation optimization. The promoters have been engineered and utility of the promoters has been validated in metabolic engineering of Y. lipolytica for producing a novel high-value product WE.

### Example 6

Engineering of Native Promoters from R. toruloides

The strength of four well-characterized promoters from R. toruloides including P_{PGK}, P_{FBA}, P_{TPI}, and P_{GPD} was measured in Y. lipolytica. As shown in FIG. 8, their activities in Y. lipolytica were very low compared with native promoter P_{TEF}. We further engineered promoter P_{G}PD by adding Y. lipolytica 16 copies of UASs, and the resulting hybrid promoter was designated P_{GPD}-UAS16. The activity of new promoter was significantly increased, and even higher than that of Y. lipolytica native promoter TEF (FIG. 8). Promoter P_{GPD}-UAS16 (SEQ ID NO: 21 modified with 16 UAS elements upstream of the promoter) was further used to replace the promoter in plasmid pYaliHex, and the new vector could be directly used for genes expression in both Y. lipolytica and R. toruloides without host-dependent optimization.

### Example 7

Development of Expression Vector pYaliHex

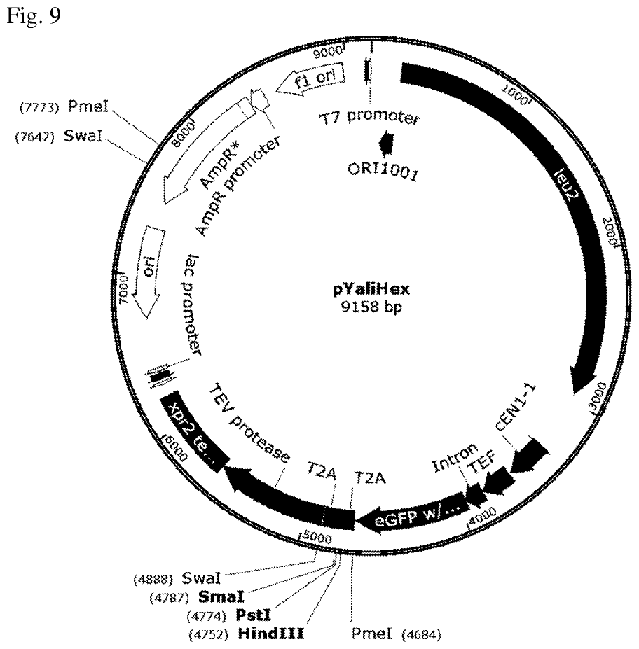

As shown in FIG. 9, plasmid pYaliHex was developed for expression of multiple genes in Y. lipolytica. In pYaliHex, there were genes encoding gfp and TEV peptidase spaced with a sequence coding for two contiguous 2A peptides. The plasmid provided multiple restriction sites such as HindIII, PstI and SmaI to clone target gene. The Ampicillin resistance gene in pYaliHex was modified to include restriction sites, PmeI and SwaI.

### Example 8

### Assembly of a Polycistronic Construct by Using Developed Vector System

As shown in FIG. 10, three steps can be carried out to clone the genes of interests and assemble a polycistronic construct consisting of T2A peptides sequence. (1) Clone of genes of interest into plasmid pYaliHex; (2) Linearization of recombinant plasmids either by PmeI (recover donor fragment) or SwaI (recover acceptor fragment); and (3) Assembly of PmeI-digested and SwaI-digested fragments by using Gibson assembly based on the homologous regions created. The resultant plasmids can be re-used as either donor or acceptor fragment to fuse with other gene or polycistronic construct because both SwaI and PmeI restriction sites are regenerated.

### Example 9

Engineering of a Cellobiose Metabolic Pathway in Y. lipolytica

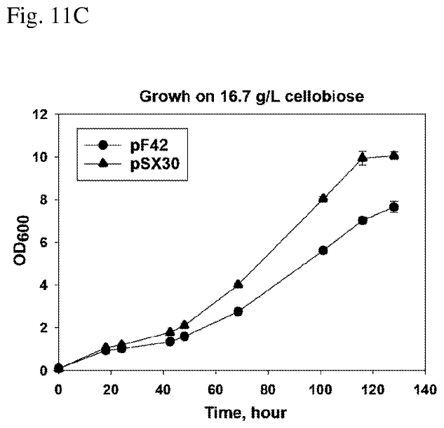

The metabolic pathway of cellobiose utilization was introduced into Y. lipolytica by heterogeneous expression of two N. crassa genes, CDT1 encoding cellodextrin transporter and BGL encoding β-glucosidase. Two methods were used to express CDT1 and BGL. The first one was co-expression of CDT1 and BGL separated with T2A peptide sequence. In the second expression vector pSX30, CDT1 and BGL was spaced with TEV cleavage site and T2A peptide sequence, and TEV encoded sequence was also included. As shown in FIGS. 11A-11C, the strain bearing the second vector (pSX30) showed better growth performance than the recombinant carrying pF2 on cellobiose under the same culture conditions. These results highlight the advantages of expression of TEV peptidase to remove the partially cleaved 2A peptides added to proteins such as CDT1 in this example, enabling the pathway to reach better performance (FIG. 11C).

### Example 10

Generation of Y. lipolytica Bearing a Disrupted Gene Encoding Protein Ku70

Various Y. lipolytica strains were isolated and reported for diverse applications such as citric acid fermentation, lipid production and environmental bioremediation. Among them, the French haploid strain W29 (ATCC 20460) is one of the most widely characterized strains. Y. lipolytica PO1f (ATCC MYA-2613), derived from strain W29, is an auxotrophic strain unable to grown on culture media lacking leucine and uracil and unable to produce extracellular protease. The genomes of Y. lipolytica W29 and PO1f have been completely sequenced. Because of the clear genetic background and auxotrophy, Y. lipolytica PO1f has been widely genetically engineered. In this embodiment, Y. lipolytica ΔKu70 was developed by knocking out the gene encoding Ku70 protein in Y. lipolytica PO1f. Deletion of Ku70 protein can facilitate the process for gene deletion and replacement by increasing the homologous recombination between the introduced gene fragments and the targeted genes in Y. lipolytica.

The parent strains Y. lipolytica W29 (ATCC 20460) and Y. lipolytica PO1f (ATCC MYA-2613) were purchased from American Type Culture Collection (ATCC). Around 2.0-kb DNA fragments homologous to upstream and downstream regions of Ku70 were sequentially cloned into plasmid pUra3loxp. After linearization of the resultant plasmid, DNA was transformed into Y. lipolytica POlf and the transformants were screened by PCR. After verification of deletion of Ku70, ura3 was removed from the strain and further the plasmid pYLCre bearing Cre recombinase gene was eliminated. In the strain, Ku70 protein was disrupted to ease the procedures for generating genes knockout and other site-specific homologous gene integration events. The advantage of Y. lipolytica ΔKu70 is that there is no need to screen for many transformants to get a desirable strain for gene deletion or site-specific gene(s) incorporation into genome.

Y. lipolytica host strain ΔKu70 is an auxotrophic strain with mutations in both leu2 and uar3 genes. Y. lipolytica ΔKu70 can grow on a complete medium such as Yeast Extract-Peptone-Dextrose (YPD) medium or minimal media supplemented with both uracil and leucine at 28-30° C. The plasmids for transformation of Y. lipolytica ΔKu70 carry either leu2 or ura3 gene, which is complementary to the corresponding deficient gene in host. The transformants can be selected for their capabilities to grow on uracil or leucine-deficient media. Until transformed, Y. lipolytica ΔKu70 is not able to grow on minimal media without either leucine or uracil.

### Example 11

Expression Vectors for Y. lipolytica

To express both heterologous and native genes in Y. lipolytica requires functional promoters to drive genes expression by using either replicable or integrative plasmids. As a critical tool in synthetic biology, promoters have been characterized and engineered Y. lipolytica. Expression vectors containing the individual and single promoters spanning the wide strength ranges are provided in this kit, and the expression vector built with a copper-inducible promoter is also included (Table 1). These expression vectors provide essential tools to fine-tune the expression of target genes. In this system, the expression cassette can be easily recovered from the vectors by digestion with the designated restriction enzymes such as XbaI/SpeI, and then can be conveniently assembled with the other one. Multiple-gene expression can be accomplished by sequential assemble of the expression cassettes containing the promoters, cloned genes, and terminators. Furthermore, the vector containing tandem 16 copies of upstream activated sequences (UAS16) from xpr2 promoter is provided to engineer the native promoters. The gene lacZ encoding β-galactosidase is provided in this kit to verify and quantify the strength of the promoter (Table 2). Finally, the expression cassettes can be further introduced into the genomes with single or high copies by cloning them into the plasmids containing the homologues sequences such as a specific target locus or partial 26s rDNA and transformation of Y. lipolytica (Table 2).

A set of expression vectors included in this kit are shown in Table 1. All the vectors listed in Table 1 contain the replication sites for both E. coli and Y. lipolytica, ampicillin resistance gene as a selection marker for E. coli, and leu2 as a selection marker for Y. lipolytica. Most of E. coli strains such as Top10, DH5α and JM109 can be used for cloning genes and propagation of the plasmids. Expression vector pYLexp2 contains the promoter tef1N, which is one of the most frequently used promoters for expression of genes in Y. lipolytica. The following maps shows the key features and their organization in pYLexp2 (FIG. 12 and Table 3). Table 2 provides a list of plasmids used herein and the primary characteristic of the plasmid. Table 2 includes the characteristics of plasmids used for the generation of a knockout strain, the plasmid for integration of gene fragment into yeast genome, and the plasmid bearing of cre encoding recombinase, developed in accordance with the present disclosure.

TABLE 1
Expression vectors developed for use in accordance with the disclosure

| | | | Replication and |
|---|---|---|---|
| Plasmid | Promoter | Terminator | Y. lipolytica marker |
| pYLexp1 | tef | xpr2 | Replicable in both E. coli and |
| | | | Y. lipolytica, leu2 |
| pYLexp2 | tef with 1^{st} intron | xpr2 | Replicable in both E. coli and |
| | (tef1N) | | Y. lipolytica, leu2 |
| pYLexp3 | fba | lip1 | Replicable in both E. coli and |
| | | | Y. lipolytica, leu2 |
| pYLexp4 | fba with 1^{st} intron | lip1 | Replicable in both E. coli and |
| | (fba1N) | | Y. lipolytica, leu2 |
| pYLexp5 | gpd | Hp2 | Replicable in both E. coli and |
| | | | Y. lipolytica, leu2 |
| pYLexp6 | gpd with 1^{st} intron | Hp2 | Replicable in both E. coli and |
| | (gpd1N) | | Y. lipolytica, leu2 |
| pYLexp7 | gpm | oct1 | Replicable in both E. coli and |
| | | | Y. lipolytica, leu2 |
| pYLexp8 | mt-2 | xpr2 | Replicable in both E. coli and |
| | | | Y. lipolytica, leu2 |

TABLE 2
Other plasmids use in accordance with the disclosure

| Plasmid | Purpose | Characteristics |
|---|---|---|
| pUra3loxp | Deletion of gene(s) in Y. | ura3 marker flanked with 34bp-loxp sites |
| | lipolytica | |
| pYLCre | Excision of ura3 flanked | Expression of ere coding recombinase in vector |
| | with loxp sites | pYLexp1 |
| pUAS16 | Increasing core promoter | Tandem 16 copies of upstream activated sequences |
| | strength | from xpr2 promoter |
| pYLInte | Integration of genes into | Contains ura3 flanked with 34 bp-loxp sites, and |
| | genome with multiple copies | partial 26s rDNA as homologous arms for genome |
| | | integration |
| pYLlacZ | Positive control for testing | Expression of lacZ encoding β-galactosidase in |
| | promoter activity | vector pYLexp1 |

TABLE 3
The generic features of the expression vector pYLexp2 (see FIG. 12)

| Feature | Description | Function |
|---|---|---|
| Pro(moter) | Promoter region from gene tef | Controls gene expression at different levels in |
| | | Y. lipolytica |
| Ter(minator) | Native transcription termination | Allows efficient transcription termination |
| Intron | 1^{st} intron from the gene tef | Usually simulates gene expression by |
| | | comparison of a promoter without intron |
| MCS | Multiple cloning site (HindIII, | Permits cloning of a gene into the |
| | PstI, Sal, PstI for pYLexp2) | expression vector |
| leu2 | Selection marker and used to | Offers a selectable marker to isolate |
| | complement leu-strain | Y. lipolytica recombinant strains |
| CEN1-1 | Centromere (CEN) cloned from | An essential element for high-efficiency |
| | Y. lipolytica chromosome | transformation of Y. lipolytica |
| ORI1001 | Replication origin (ORI) isolated | Enables a plasmid to replicate independently |
| | from Y. lipolytica chromosome | of the yeast chromosome |
| Ori | pBR322 origin of E. coli | Permits replication and high-copy existence in |
| | replication | E. coli |
| Amp^{R} | Ampicillin resistance gene | Provides a selection marker of plasmids in E. |
| | | coli |

### Example 12

Transformation of Y. lipolytica with Expression Vectors

Plasmid DNA for Y. lipolytica transformation can be prepared with routine molecular biology techniques. Without linearization, the plasmids derived from expression vectors provided in this kit (Table 2) can be used to directly transform Y. lipolytica. Although various protocols and methods have been developed for genetic transformation of Y. lipolytica, Frozen-EZ Yeast Transformation II Kit (Zymo Research, Irvine, Calif., U.S.) is recommend for transformation by following the manufacturer's guidelines due to the convenience and efficiency. The yeast transformants can be plated on agar plates of synthetic media without leucine consisting of 20 g/L glucose, 6.7 g/L yeast nitrogen base (YNB) without amino acid and with ammonium sulfate (US Biologicals), supplemented with 2.0 g/L of complete supplement of amino acids lacking leucine (US Biologicals). After culturing for 3 days at 28-30° C., the colonies can be visible and ready to be picked up from agar plates. Similarly, synthetic liquid media without leucine can be used to culture the recombinants.

### Example 13

Deletion and Integration of Genes in Y. lipolytica

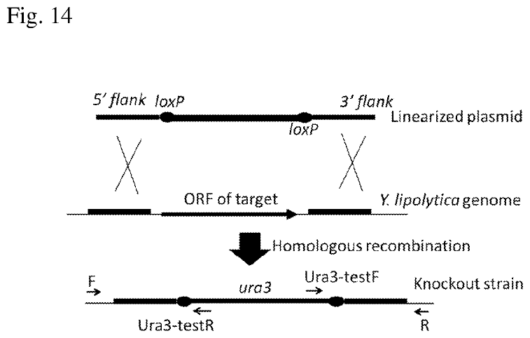

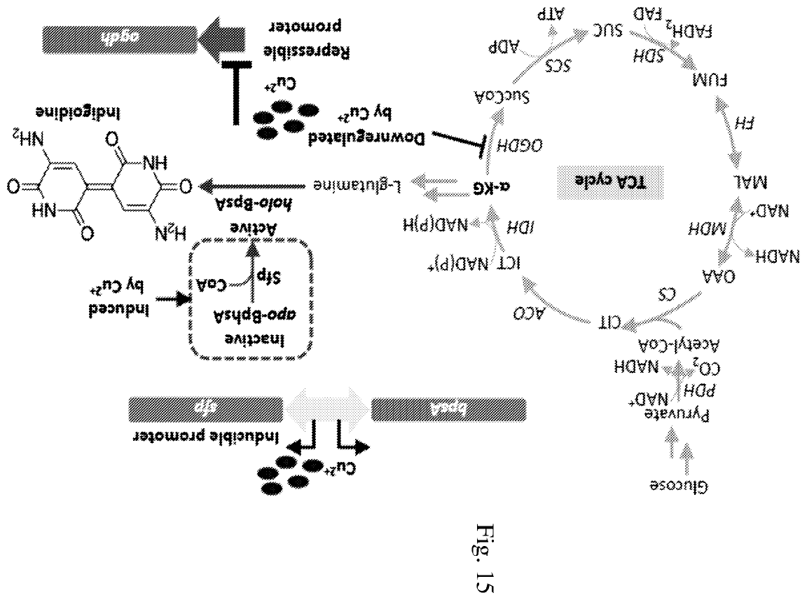

Deletion of a gene can be used to study gene function and block a metabolic pathway. Generation of a gene knockout of Y. lipolytica involves developing a plasmid containing the upstream and downstream homology arms and a selectable marker (e.g., uar3) to replace the target gene to be knockout. This plasmid is used to transform Y. lipolytica, optionally using the linearized plasmid, and verification of gene deletion. In this embodiment, ura3 is flanked with 34-bp loxp sites, and thus the selectable marker can be removed by expression of cre encoding recombinase after confirmation of the desired recombination event (see FIG. 14). Through this iterative gene integration and marker curation process, combinational genes knockout of Y. lipolytica can be created. Furthermore, expression cassette(s) can be cloned into the plasmids containing the homologous regions for integration of them into the site-specific sites. Generally, the presence of the genes is more stable in yeast genome than the existence of the genes cloned in a replicable vector. Finally, the gene fragments can be integrated into the genome with high-copy number through 26s rDNA integration.

One set of procedures for deletion of a targeted gene in Y. lipolytica are provided below:

Step 1: Generate Disruption Plasmid and Transform Yeast with Linearized Plasmid

Around 1-kb homologous 5′ flank and 3′ flank of a targeted gene (optionally 26s rDNA sequences) can be cloned into restriction sites of ApaI/XbaI and SpeI/NdeI in plasmid pUra3loxp, respectively (plasmid map can be found in FIG. 13). Linearization of the resultant plasmid can be carried out by single digestion with ApaI or NdeI without disrupting the cloned fragments, and then the recovered DNA can be used to transform Y. lipolytica ΔKu70. After transformation using Frozen-EZ Yeast Transformation II Kit (Zymo Research, Irvine, Calif., U.S.), the yeast transformants can be grown on agar plates of synthetic media consisting of 20 g/L glucose, 6.7 g/L YNB (US Biologicals), and supplement of amino acids lacking uracil (US Biologicals) at 28-30° C.

### Step 2: Verify Gene Knockout by PCR Diagnosis

After 2-3 days, single colonies are picked, and further cultured in YPD broth at 28-30° C. At the same time, the colonies can be replicated on YPD agar plates. Usually, 6 colonies are enough to get a strain with a disrupted gene. After cultivating for 1-2 days, 1.0 ml of yeast culture is used for extraction of genomic DNA. Although there are different approaches and kits available for extraction of genomic DNA from yeast cells, the following procedures have been validated as an efficient, fast and cheap method to get relatively high-quality genomic DNA suitable for PCR.

1). Harvest and resuspend cells in 500 μl lysis solution consisting of 200 mM Lithium Acetate and 1% SDS;

2). Incubate for 20 minutes at 70° C.;

3). Add the same volume of chloroform: isoamyl alcohol (24:1), vortex and centrifuge;

4). Collect the aqueous phase and add two volumes of 96-100% ethanol;

5). Keep at −20° C. in the freezer for at least 2 hours, and centrifuge to get precipitated DNA;

6). Wash DNA pellet with 1 ml 70% ethanol;

7). Dissolve pellet in 30 μl of H₂O or TE buffer;

8). Use 0.5 μl of DNA solution as template for PCR in 20-ul reaction mixture;

The primers of ura3-testF, ura3-testR and two primers (F and R) localized outside of 5′ and 3′ flanks are designed to generate PCR products to verify the crossover event. The sequences (5′ to 3′) of the primers and ura-testF and ura3-testR used in this embodiment are:

ura3-testF:
(SEQ ID NO: 18)
TCCTGGAGGCAGAAGAACTT;
ura3-testR:
(SEQ ID NO: 19)
AGCCCTTCTGACTCACGTAT;

However, other suitable primers can be designed based on the sequence of uar3 marker to perform a similar function. The gene knockout is verified by performing agarose gel electrophoresis to check the size of PCR products.

### Step3: Marker Rescue by Expression of Recombinase

The following steps can be used to remove ura3 marker in the knockout strain.

1). Culture the single colony of identified knockout strain in YPD broth at 28-30° C. After harvesting the cells, the strain is transformed with plasmid pYLCre bearing a nucleic acid encoding a cre recombinase. The yeast transformants are plated on agar plates of synthetic YNB media without leucine;

2). Pick the transformants from agar plates of synthetic YNB media without leucine, and inoculate them into YPD broth;

3). Streak the overnight culture on YPD agar plates to get single colonies, and incubate for 1 day at 28-30° C.;

4). Pick up the single colonies (usually 10 strains are enough) and replica them onto two synthetic media plates: selective for uar⁻ and on leu⁻, as well as YPD agar plates. Cells that cannot grow on the medium without uracil does not have ura3, and the plasmid pYLCre is lost in the cells that cannot grow on the medium without leucine. To verify marker loss, PCR can be carried out with the appropriate primers. The strain without uar3 gene and without plasmid pYLCre can be used for gene deletion in the next round.

1. A transcription element comprising
a promoter; and
a polylinker,
wherein said promoter comprises a nucleic acid sequence selected from the group consisting of SEQ ID NO: 1 (P_{MT-1}), SEQ ID NO: 2 (P_{MT-2}), SEQ ID NO: 3 (P_{MT-3}), SEQ ID NO: 4 (P_{MT-4}), SEQ ID NO: 5 (P_{MT-5}), SEQ ID NO: 6 (P_{MT-6}), SEQ ID NO: 7 (PTurzi), SEQ ID NO: 8 (PMET3), SEQ ID NO: 9 (P_{SER1}), SEQ ID NO: 10 (P_{CTR1}), and SEQ ID NO: 11 (PcTR2) or a nucleic acid sequence having at least 95% sequence identity with a nucleic acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11, and further wherein said polylinker is operably linked to said promoter sequence.

2. The transcription element of claim 1 wherein said promoter sequence comprises a sequence selected from the group consisting of SEQ ID NO: 1, SEQ ID NO: 2, SEQ ID NO: 3, SEQ ID NO: 4, SEQ ID NO: 5 and SEQ ID NO: 6, optionally wherein said promoter sequence comprises of a sequence selected from the group consisting of SEQ ID NO: 2 and SEQ ID NO: 6, optionally wherein the promoter comprises SEQ ID NO: 1 and SEQ ID NO: 2.

3. The transcription element of claim 2 further comprising 1 to 16 UAS sequences operably linked to said promoter sequence, optionally wherein each of said UAS sequence are identical and comprises the sequence of SEQ ID NO: 12.

4. The transcription element of claim 1 further comprising a 2A polypeptide coding nucleic acid sequence located downstream from said polylinker, optionally wherein the encoded 2A peptide has the sequence of GSGEGRGSLLTCGDVEENPGP (SEQ ID NO: 13) or GSGATNFSLLKQAGDVEENPGP (SEQ ID NO: 14).

5. The transcription element of claim 4 wherein the 2A polypeptide coding nucleic acid sequence comprises the sequence having 99% sequence identity to SEQ ID NO: 15.

6. The transcription element of claim 4 wherein said transcription element comprises a plurality of 2A polypeptide coding nucleic acid sequences, wherein each of said plurality of 2A polypeptide coding nucleic acid sequences is each proceeded by at least one restriction enzyme cleavage site that is unique to the transcription element.

7. The transcription element of claim 6 further comprising a nucleic acid encoding a TEV peptidase.

8. The transcription element of claim 1 further comprising a 1st intron from the gene tef positioned between said promoter and the polylinker.

9. The transcription element of claim 1 formed as a plasmid.

10. The transcription element of claim 2 wherein said promoter is flanked on each end of the promoter sequence with a polylinker sequence.

11. The transcription element of claim 1 further comprising a selectable marker, optionally wherein the selectable marker is an auxotrophic marker, optionally wherein the auxotrophic marker is leu2.

12. The transcription element of claim 1 further comprising an antibiotic resistance gene as a selectable marker.

13. The transcription element of claim 1 further comprising a replication region for Y. lipolytica.

14. The transcription element claim 1 further comprising a replication region for E. coli.

15. The transcription element of claim 1 wherein said promoter is operably linked to a heterologous coding sequence.

16. A Yarrowia lipolytica or Rhodotorula toruloides host cell comprising the nucleic acid of claim 15, optionally wherein the host cell is a Ku70-deleted strain.

17. A method of simultaneously inducing the expression of two gene products by induction of a single control element, said method comprising
providing a host cell that comprises a Cu²⁺-inducible promoter operably linked to both a first gene on the plus strand of said promoter and a second gene on the negative strand, wherein said promoter comprises a nucleic acid sequence selected from the group consisting of SEQ ID NO: 1, SEQ ID NO: 2, SEQ ID NO: 3, SEQ ID NO: 4, SEQ ID NO: 5, and SEQ ID NO: 6, or a sequence having at least 95% sequence identity to a nucleic acid sequence selected from the group consisting of SEQ ID NO: 1, SEQ ID NO: 2, SEQ ID NO: 3, SEQ ID NO: 4, SEQ ID NO: 5, and SEQ ID NO: 6;
contacting said host cell with an amount of Cu²⁺ that induces bidirectional transcription from said promoter to induce expression of said first and second genes.

18. The method of claim 17 wherein a plurality of genes are operably linked to said promoter in a tandem array wherein a 2A polypeptide coding sequence is located at the 3′ terminus of all but the last of said plurality of genes.

19. The method of claim 17 further comprising the step of decreasing the expression of an endogenous gene, wherein
a repressible heterologous promoter operably linked to said endogenous gene is inhibited by contacting the host cell with the inhibitory agent, optionally wherein the repressible promoter comprises of a sequence selected from the group consisting of SEQ ID NO: 10 (CTR1) and SEQ ID NO: 11 (CTR2).

20. A kit comprising
an inducible promoter sequence selected from the group consisting of SEQ ID NO: 1, SEQ ID NO: 2, SEQ ID NO: 3, SEQ ID NO: 4, SEQ ID NO: 5, and SEQ ID NO: 6, optionally formed as a first plasmid; and
a second plasmid wherein said second plasmid comprises
a first and second pair of 34-bp loxp sites flanking a nucleic acid sequence encoding a selectable marker gene;
a first restriction site located upstream of said first loxp site; and
a second restriction site located downstream of said second loxp site, wherein said first and second restriction sites are different from each other and are unique to said second plasmid.

21.-28. (canceled)
